(19) United States
(12) Patent Application Publication
(10) Pub. No.: US 2005/0048547 A1
(43) Pub. Date: Mar. 3, 2005

(54) Classification of disease states using mass spectrometry data

(76) Inventors: Hongyu Zhao, Guilford, CT (US); Kenneth R. Williams, North Haven, CT (US); Baolin Wu, Minneapolis, MN (US); Kathryn Stone, Westbrook, CT (US); Walter McMurray, Madison, CT (US); Thomas Abbott, Branford, CT (US)

Correspondence Address: WELSH & FLAXMAN LLC, 2450 CRYSTAL DRIVE, SUITE 112, ARLINGTON, VA, 22202, US

(21) Appl. No.: 10/893,434

(22) Filed: Jul. 19, 2004

Related U.S. Application Data
(60) Provisional application No. 60/488,371, filed on Jul. 17, 2003.

(51) Int. Cl. C12Q 1/68; G06F 19/00; G01N 33/48; G01N 33/50

(52) U.S. Cl. 435/6; 702/20

(57) ABSTRACT

A method for identification of biological characteristics is achieved by collecting a data set relating to individuals having known biological characteristics and analyzing the data set to identify biomarkers potentially relating to selected biological state classes. A system for identification of biological characteristics is also provided. A methodology is also provided for utilizing mass spectroscopy data to identify peptide and protein biomarkers that can be used to optimally discriminate experimental from control samples—where the experimental samples may, for instance, be derived from patients with various diseases such as ovarian cancer.

### CROSS REFERENCE TO RELATED APPLICATIONS

This application is based upon U.S. Provisional Patent Application Ser. No. 60/488,371, filed Jul. 17, 2003, and entitled “Classification of Disease States Using Mass Spectrometry Data”.

### BACKGROUND OF THE INVENTION

1. Field of the Invention

The invention relates to a comprehensive statistical, computational, and visualization approach to identifying the naturally occurring forms of peptide and protein disease biomarkers from raw data collected from mass spectrometric (MS) instruments. More particularly, the invention employs background subtraction, spectrum alignment (registration), peak identification, normalization, and outlier detection. The disease biomarker identification uses a customized Random Forest algorithm to search for features that show distinct patterns among different classes of samples.

2. Description of the Prior Art

DNA microarray analysis offers a breakthrough and massively parallel approach to genome-wide expression analysis that, for many purposes, is unfortunately directed at the wrong biological molecule. Differential rates of translation of mRNAs into protein and differential rates of protein degradation in vivo are two factors that confound the extrapolation of mRNA to protein expression profiles. For instance, Gygi et al. estimate the correlation between protein and mRNA abundance for yeast is only 0.4. Gygi, S. P., Rochon, Y, Franza, B. P., and Aebersold, R., Correlation between protein and mRNA abundance in yeast, Mol. Cell. Biol. 19, 1720-1730 (1999). They found yeast genes with similar mRNA levels that had protein levels that differed by 20-fold. Conversely, they found invariant, steady-state levels of proteins which had mRNA levels that varied by 30-fold, similar to the >10-fold range observed by Futcher et al. Futcher, B., Latte, G. I., Monardo, P., McLaughlin, C. S., and Garrels, J. I., A sampling of the yeast proteome, Mol. Cell. Biol. 19, 7357-7368 (1999). Additionally, microarray analysis is unable to detect, identify or quantify post-translational protein modifications which often play a key role in modulating protein function. Protein expression analysis offers a potentially large advantage in that it measures the level of the biological effector protein molecule, not just that of its message.

Proteomics is an integral part of the process of understanding biological systems, pursuing drug discovery, and uncovering disease mechanisms. The identification of protein biomarkers correlating with specific diseases will permit earlier detection of diseases, allow more accurate classification of diseases based upon protein expression rather than just clinical and histological data, provide more effective means for following the course of disease and facilitate the identification of proteins involved in the disease process for improving the understanding of diseases and leading to new and more effective treatments.

Because of their importance and the very high level of variability and complexity, the analysis of protein expression is as potentially exciting as it is a challenging task in life science research. Proteomics. Science 294, 5549, 2074-2085 (2001). Comparative profiling of protein extracts from normal versus experimental cells and tissues enables us to potentially discover novel proteins that play important roles in disease pathology, response to stimuli, and developmental regulation. However, to conduct massively parallel analysis of thousands of proteins, over a large number of samples, in a reproducible manner so that logical decisions can be made based on qualitative and quantitative differences in protein content is an extremely challenging endeavor.

The prior art does not make it currently possible to carryout a massively parallel, quantitative analysis of the level of expression of tens of thousands of proteins, over a large number of samples, in a reproducible manner that approaches that of DNA microarray technology for mRNA expression. Two approaches that have been used to quantitatively and simultaneously profile approximately 500-1,000 proteins are isotope coded affinity tags (ICAT) coupled with liquid chromatography/mass spectrometry (LC/MS) and 2D differential (fluorescence) gel electrophoresis (DIGE). Han, D. K., Eng, J. M., Zhou, H, and Aebersold, R., Quantitative profiling of differentiation induced microsomal proteins using isotope-coded affinity tags and mass spectrometry, Nature Biotechnology 19, 946-951 (2001); Zhou, G., Li, F L, DeCamp, D., Chen, S., Shu, H, Gong, Y., Flaig, M., Gillespie, J W., Hu N., Taylor, P R, Emmert-Buck, M R., Liotta, L A., Petricoin, E F., Zhao, Y., 2D differential in-gel electrophoresis for the identification of esophageal scans cell cancer-specific protein markers, Molecular & Cellular Proteomics, 1(2), 117-24 (2002). The ICAT study by Han et al compared protein expression in microsomal fractions of control versus in vitro differentiated human myeloid leukemia cells. In this study, the tryptic digest of the microsomal protein extract was separated into 30 fractions via cation exchange HPLC Each of these 30 fractions was then subjected to avidin affinity chromatography followed by LC/MS/MS. During this study 25,892 individual MS/MS spectra were analyzed and subjected to database searching. More than 5,000 cysteine-containing peptides were identified with this massive effort which resulted in quantifying the relative level of expression of 491 proteins (which were also identified) in only one control versus experimental sample. In comparison, in the DIGE study of Zhou et al., a single 2D gel containing a protein extract from laser capture microdissected esophageal cancer cells that was labeled with Cy5 and a similar extract from normal cells that was labeled with Cy3 resulted in quantifying the relative (spot volume) intensities of 1,264 fluorescent spots.

Both the ICAT/LC-MS and DIGE approaches to protein profiling share the commonality of trying to quantify the relative level of expression of as many proteins as possible to uncover the (perhaps) 5%, or so, of proteins which are the most substantially up or down-regulated. With this in mind, and as will be discussed below in the Description of the Preferred Embodiment, the peptide disease biomarker approach employed in accordance with the present invention provides a novel approach in that from the beginning it is directed at finding the peptides that are of the most interest; that is, the 5-40 or so peptides whose intensities can best differentiate all control from experimental spectra. And, in most instances, it is not necessary that the peptide biomarker peaks be completely resolved as it is possible to search at the level of individual m/z (mass charge ratio) versus intensity data points. In effect, peptide disease biomarker discovery in accordance with the present invention provides a “short-cut” approach to protein profiling that enables large numbers of raw and extremely complex spectra to be effectively analyzed, thus obviating challenges resulting from biological diversity within the control and experimental samples.

The relative simplicity of the peptide disease biomarker approach, the potential importance of the resulting biomarkers, and the availability of a commercial laser desorption ionization time-of-flight MS platform that provides a “single step” approach for desalting and spotting biological samples accounts for the rapidly increasing number of researchers using this technology. Surface enhanced laser desorption ionization time-of-flight mass spectrometry (SELDI-TOF-MS) involves the use of a 10 mm×80 mm chip having eight or sixteen 2 mm spots comprised of specific chromatographic surfaces (e.g., anionic, cationic, hydrophobic, hydrophilic, metal, etc). Issaq, H. J., Veenstra, T. D., Conrads, T. P., Felschow, D. Breakthroughs and Views; The SELDI-TOF MS Approach to Proteomics: Protein Profiling and Biomarker Identification, Biochemical and Biophysical, Research Communications 292, 587-592 (2002). After spotting a few microliters of serum or other biological sample onto the chip surface, desalting is accomplished via washing with water prior to adding and then drying onto the target a solution of an energy absorbing reagent like α-cyano-4-hydroxy-cinnamic acid (that is, the “matrix” in conventional matrix assisted laser desorption ionization mass spectrometry (MALDI-MS)).

One of the reports that has helped spur more widespread interest in SELDI based detection of peptide/protein disease biomarkers is the ovarian cancer study of Petricoin et al. In this study, SELDI-MS analysis of sera from 50 control and 50 case samples from patients with ovarian cancer resulted in identifying 5 peptide biomarkers that ranged in size from 534 to 2,465 Da. Petricoin, E. F., Ardekani, A M., Hitt, B. A, Levine, P. J., Fusaro, V. A., Steinberg, S. M., Mills, G. B., Simine, C, Fishman, D. A., Kohn, E. C., and Liotta, L. A., Use of proteomic patterns in serum to identify ovarian cancer, The Lancet 359, 572-77 (2002); U.S. Patent Application Publication No. 2003/0004402 to Hitt et al. The pattern formed by these markers was then used to correctly classify all 50 ovarian cancer samples in a masked set of serum samples from 116 patients who included 50 patients with ovarian cancer and 66 unaffected women or those with non-malignant disorders. Of the latter samples, 63 were correctly recognized as not being from cancer patients thus providing 100% sensitivity (50/50) for detecting cancer, 95% specificity (63/66) for detecting controls, and a positive predictive value of 94% (50/53). That is, if the 5 peptide “ovarian cancer” biomarker pattern was identified in the sample, there was a 94% probability that the patient indeed has ovarian cancer.

Similar promising results have been reported recently in two other reasonably large scale studies of serum samples from breast and prostrate cancer patients. In the case of breast cancer, Li et al. identified three biomarkers (m/z=4,300, 8,100 and 8,900), which together demonstrated a sensitivity of 93% for 103 breast cancer patients and a specificity of 91% for 66 controls that included 41 healthy women and 25 patients with benign breast diseases. Li, J., Zhang, Z., Rosenzweig, J., Wang, Y. Y., Chan, D. W., Proteomics and Bioinformatics Approaches for Identification of Serum Biomarkers to Detect Breast Cancer, Clinical Chemistry 48:8, 1296-1304 (2002). In the case of prostrate cancer, Adam et al identified nine m/z between 4,475 and 9,656 Da that demonstrated a sensitivity of 83%, a specificity of 97% and a positive predictive value of 96% based on the analysis of serum samples from 167 patients with prostrate cancer and 159 patients who were either healthy or had benign prostrate hyperplasia. Adam, B. L., Vlahou, A, Semmes, J. O., Wright, Jr. G. L., Proteomic approaches to biomarker discovery in prostate and bladder cancers, Proteomics 1, 1264-1270 (2001). Finally, Vlahou et al. used a similar SELDI-MS approach to identify two biomarkers m/z=3,300/3,400 and 9,500) and a protein “cluster” (which had m/z ranging from 85,000 to 92,000) in urine which together provided a sensitivity of 87% for detecting transitional cell carcinoma of the bladder. In this latter study, a total of 94 urine samples were analyzed and the corresponding specificity was 66% and the positive predictive value was 54%. Vlahou, A., Schellhammer, P. F., Mendrinos, S., Patel, K., Kondylis, F. I., Gong, L., Nasim, S., Wright, Jr. G. L., Development of a Novel Proteomic Approach for the Detection of Transitional Cell Carcinoma of the Bladder in Urine, American Journal Pathology 158:4, 1491-1502 (2001). Taken together, these studies certainly seem sufficiently promising to warrant larger scale studies and extension of similar approaches to the study of other cancers and disease states.

Despite some of the results discussed above, traditional statistical methods for classification are not optimal or even appropriate for biomarker identification using mass spectrometry data. As the data is very high dimensional, dimension reduction is necessary before using these methods for biomarker identification. Principal component analysis (PCA) is a common method for dimension reduction. PCA is based on SVD (singular value decomposition), and has been applied in microarray data analysis. However, the interpretation of PCA is not straightforward. In the microarray data analysis context, Alter et al. use ‘Eigengenes’ to interpret the results of SVD analysis, however, this is not intuitive. Alter, O., Brown, P. O., and Botstein, D. Singular value decomposition for genome-wide expression data processing and modeling, PNA S 97, 18 (2000), 10101-10106. Some traditional discriminant analysis techniques, e.g. LDA (linear discriminant analysis) and QDA (quadratic discriminant analysis), are model-dependent. Fisher R. A. (1936). The use of multiple measurements in taxonomic problems. Annal of Eugenics, 7:179-188. They make strong assumptions about the underlying data distribution, which may rarely hold for complex data. As a result, they can be biased for large complex datasets. On the other hand, model independent methods, e.g. CART (classification and regression trees), maybe highly variable due to the high dimensionality of the mass spectrometry data. Breiman L., Friedman, J. H., Olshen, K A. and Stone, C J. Classification and Regression Trees (1983).

As the previous discussion shows, mass spectrometry (MS) is increasingly being used for rapid identification and characterization of protein populations. There have been tremendous research efforts recently trying to utilize mass spectrometry technology to build molecular diagnosis and prognosis tools for cancers. Petricoin et al.; Adam et al.; Li et al. Most of the papers have claimed ≧90% sensitivity and specificity using a subset of selected biomarkers; some of them even report achieving perfect classification. Zhu, W., Wang, X., Ma, Y., Rao, M., Glib J., and Kovach, J. S., Detection of cancer specific markers amid massive mass spectral data, PNAS 100, 25, 14666-14671 (2003). But upon our closer inspection of these studies, many of the identified biomarkers actually appear to arise from background noise, which suggests some systematic bias from non-biological variation in the dataset. Additionally, all these studies reflect the neglected importance of data preprocessing and of appropriately interpreting large mass spectrometry datasets. Another commonly neglected fact is the correct way of using cross-validation.

As discussed in Ambroise et al., it is important to do an external cross-validation, whereby at each stage of the validation process one must not use any information from the testing set to build the classifier from the training set. Ambroise, C and McLachlan, G. J., Selection bias in gene extraction on the basis of microarray gene-expression data, PNAS 99, 10 (2002), 6562-6566. Internal cross-validation is used in most current disease biomarker mass spectrometry studies, whereby the selection of biomarkers has utilized information from all the samples, which will significantly (e.g., see below) under-estimate classification error.

We previously studied the relative performance of popular classification methods in the context of a mass spectrometry ovarian cancer dataset and published our results. Wu, B., Abbott, T., Fishman, D., McMurray, W., Mor, G., Stone, K., Ward, D., Williams, K., and Zhao, H, Comparison of statistical methods for classification of ovarian cancer using mass spectrometry data, Bioinformatics 19, 13, 1636-1643 (2003a).

Our re-examination of data used in the Petricoin et al. study illustrates the importance of visualization tools and some of the unique challenges of analyzing mass spectrometry data sets. Petricoin et al. employed Genetic Algorithms and Self-Organizing Maps to analyze SELDI spectra obtained on serum to identify peptide biomarkers to distinguish ovarian cancer patients from normal individuals. David E. Goldberg, Genetic Algorithms in Search, Optimization, and Machine Learning, Addison-Wesley Pub Co. (1989); Teuvo Kohonen, T. S. Huang, M. R. Schroeder, Self-Organizing Maps, Springer-Verlag (2000). However, visualization of the m/z regions around each of the 5 ovarian cancer biomarkers identified in their study suggests that many of their biomarkers may derive from variations in background noise (see FIG. 2) rather than from peptide ionization. With so many (typically >90,000 in the present study using only reflectron acquired data) data points being analyzed in each spectrum there is a reasonable probability that at least a few of theses points will (by chance alone) be able to “differentiate” cases from controls in the training sets. Obviously, however, the latter will have little subsequent value. FIG. 3, which shows the 800-3500 m/z region for two representative normal and ovarian cancer serum spectra, demonstrates the comparatively low signal/noise ratio of data in this region that was obtained by the instrumentation used by Petricoin et al. As was shown in FIG. 1, a much higher signal/noise ratio can be obtained over this region from desalted serum that is analyzed on a conventional Micromass MALDI-MS instrument equipped with a reflectron analyzer. Obviously, in this instance, the ability to easily visualize the m/z regions around biomarkers that have been selected by sophisticated statistical approaches adds substantial value to the overall analysis. In the following section, we describe robust statistical methods that address the issues discussed above, and then apply these methods to analyze on a conventional MALDI mass spectrometer an ovarian cancer data set similar to that analyzed by Petricoin et al.

More particularly, in the Petricoin et al study, SELDI-MS analysis of serum from 50 control and 50 case samples from patients with ovarian cancer resulted in identifying 5 peptide biomarkers that ranged in size from 534 to 2,465 Da. The pattern formed by these biomarkers was then used to correctly classify all 50 ovarian cancer samples in a masked set of serum samples from 116 patients who included 50 ovarian cancer patients and 66 unaffected women or those with non-malignant disorders. Of the latter samples, 63 were correctly recognized as not being from cancer patients—thus providing 100% sensitivity (50/50) for detecting cancer, 95% specificity (63/66) for detecting controls, and a positive predictive value of 94% (50/53) for this population. That is, if the 5 peptide “ovarian cancer” biomarker pattern was identified in the sample, there was a 94% probability that the patient indeed has ovarian cancer. Although similar promising results have been reported recently in other reasonably large-scale studies of serum samples from breast and prostrate cancer patients (Li, J., Zhang, Z., Rosenzweig, J., Wang, Y. Y., Chan, D. W., Proteomics and Bioinformatics Approach for Identification of Serum Biomarkers to Detect Breast Cancer, Clinical Chemistry 48:8, 1296-1304 (2002); Bao-Ling Adam, Yisheng Qu, John W. Davis, Michael D. Ward, Mary Ann Clements, Lisa R Cazares, O. John Semmes, Paul F. Schellhammer, Yutaka Yasui, Ziding Feng, and George L. Wright, Jr., Serum Protein Fingerprinting Coupled with a Pattern-matching Algorithm Distinguishes Prostate Cancer from Benign Prostate Hyperplasia and Healthy Men, Cancer Res. 62: 3609-3614 (2002)), we would like to raise two concerns about the Petricoin et al study. The first is an issue that was raised by Rockville and others and that is the very high positive predictive value (PPV) of 94% reported by Petricoin et al applies only to their artificial population of 116 patients, 50 of whom had ovarian cancer. When their estimates of sensitivity (100%) and specificity (95%) are applied to an average population of post-menopausal women with an incidence of ovarian cancer of 50 per 100,000, the PPV is reduced to a clinically insignificant value of only 1%. Rockhill, B, Proteomics patterns in serum and identification of ovarian cancer, The Lancet 360, 169-170 (2002). The second caution with regard to the Petricoin et al. study is that (as shown below) closer examination of the mass spectra around their “biomarkers” suggests strongly that the latter do not arise from biologically significant peptides.

The deceptively straightforward approaches now being used (often by non-mass spectroscopists) to uncover naturally occurring peptide and protein biomarkers of disease hold enormous promise for bringing the power of mass spectrometry to bear on the challenge of protein profiling the large numbers of samples needed to obviate biological diversity. However, challenging statistical issues remain that often have not been well addressed in the existing work. The present method and system provides a straightforward methodology that allows for application of peptide disease biomarker discovery on a far wider range of mass spectrometric instrumentation. The present method and system provides a refined statistical method to address a range of important issues including background subtraction, peak identification, and normalization of spectra; and then, we introduce visualization tools, and a new algorithmic approach to uncovering peptide and protein biomarkers of disease. Using previously published and newly acquired data on serum from control versus ovarian cancer patients, the present method provides practical guidelines for using this technology and suggest how it might be applied in the future to the far more daunting challenge of analyzing multiple spectra/sample and of proteome profiling. Our study supports the superior performance of the Random Forest approach. We use Random Forest to estimate the unbiased classification error for our ovarian cancer mass spectrometry data. In the meantime we also empirically evaluate the impacts of a number of selected biomarkers and the sample size on classification error. Our analysis framework will provide a general guideline for the practice of utilizing mass spectrometry for cancer and other disease molecular diagnosis and prognosis.

As such, the present method and system provide an advanced mechanism whereby various diseases maybe identified based upon the analysis of irregularities found in protein analysis. In accordance with the present invention, we provide an improved method for identifying various biomarkers, for example, those associated with ovarian cancer. In doing so, the present invention overcomes some of the challenges of statistically analyzing MALDI-MS datasets that inherently are noisy and have a very high ratio of variables (ie, m/z vs. intensity data points) to samples. The present invention also demonstrates how the serum disease biomarker discovery approach can be extended to more commonly available “MALDI-MS” instrument platforms, customizes a Random Forest algorithm for identifying biomarkers, and suggests how the disease biomarker strategy might be extended to even more sophisticated mass spectrometry platforms, to the analysis of multiple spectra/sample, and to proteome-level profiling.

### SUMMARY OF THE INVENTION

It is, therefore, an object of the present invention to provide a method for identification of biological characteristics that is achieved by collecting a data set relating to individuals having known biological characteristics and analyzing the data set to identify biomarkers potentially relating to selected biological state classes.

It is also an object of the present invention to provide a system for identification of biological characteristics which includes means for collecting a data set relating to individuals having known biological characteristics and means for classifying the data set to identify biomarkers potentially relating to selected biological state classes.

It is another object of the present invention to provide methodology for utilizing mass spectroscopy data to identify peptide and protein biomarkers that can be used to optimally discriminate experimental from control samples—where the experimental samples may, for instance, be derived from patients with various diseases such as ovarian cancer.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 shows mass spectrometry spectra (obtained with a reflectron analyzer on a Micromass M@LDI-R mass spectrometer) for 4 selected samples. Sample 1 & 2 are normal subjects, sample 3 & 4 are cancer subjects. The x-axis is the mass-to-charge (m/z) measurements that range from 800 Da to 3500 Da and the y-axis is the measured raw intensities that have a wide dynamic range for different samples. Viewing these spectra (e.g., spectra 2-4) one can also see the characteristic decreasing trend in the measured intensities obtained with a reflectron analyzer as the m/z ratio increases.

FIG. 2 shows regions around 5 identified biomarkers from the Petricoin et al. study. There are a total of 50 case samples and 50 control samples. Instead of overlaying 100 samples in each plot, we plotted several quantiles for the case/control group. In the plot, q0.25 is the 25^{th} percentile, and q0.75 is the 75^{th} percentile. We plotted 50 measurements around each biomarker. One can clearly see that at least 3 of these 5 biomarkers are very likely to arise from background noise as there do not appear to be any discernable peptide peaks at positions corresponding to the 534,989 and 2464 biomarkers. In addition, Petricoin et al. attempt to identify biomarkers within the range of m/z<650 Da where those skilled in the art will appreciate that results are highly unreliable due to overwhelming noise within this range. The latter results from the chemical matrix that must be added to the samples to induce peptide and protein ionization.

FIG. 2.1 illustrate SELDI mass spectrometry spectra for 4 selected samples from Petricoin et al. within the range extending from 800 Da to 3500 Da. Samples 1 & 2 are normal subjects and samples 3 & 4 are cancer subjects. The y-axis is the normalized intensity using the method described in Petricoin et al. Compared to FIG. 1 from the Micromass M@LDI-R instrument, these SELDI-MS spectra have considerably less resolution.

FIG. 3 shows the estimated background for 4 previously selected samples. Due to the wide dynamic range of the intensity measurements, we take the logarithm of the intensities to reduce the numerical variation. After taking the log we estimate the background for each sample and subtract these background intensities. In terms of the raw intensities, we are actually dividing each sample by our estimated background. In this log scale plot, the decreasing trend of intensity with increasing m/z is more obvious.

FIG. 4 shows the reproducibility of spectra obtained from individual MALDI-Ms laser shots. This plot compares the coefficient of variation for 130 selected peaks from the serum of one subject across 40 individual laser shots before/after taking the log transformation. We can clearly see that taking the log has substantially reduced the noise level.

FIGS. 5.1, 5.2 and 5.3 plot the mean intensities of manually processed samples vs. the mean intensities of robotically processed samples.

FIG. 6 shows case/control median plots for 175 samples without any preprocessing. The first two panels are the median intensities across all cases/controls. The third panel shows the difference of case/control medians.

FIG. 7 shows case/control median plots for 175 samples after all preprocessing. The first two panels show the median intensities across all cases/controls. The third panel shows the difference of case/control medians.

FIG. 8 shows the distribution of peaks for all samples at each point.

FIG. 9 shows the ranking measures of selected peaks.

FIG. 10 shows five-fold cross-validation estimation of Err(N, M) for the ovarian cancer data. The left panel is based on reflectron analyzer data only while the right panel is based on the reflectron+linear analyzer data—where the latter two spectra have been joined together.

FIG. 11 shows classification error extrapolation for reflectron+linear analyzer data.

FIGS. 12 to 15 show local exploration of identified biomarkers.

FIG. 16 is a schematic of the system employed in accordance with the present invention.

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limited, but merely as the basis for the claims and as a basis for teaching one skilled in the art how to make and/or use the invention.

The present invention provides a method and system for the identification of biological characteristics. Briefly, the method is achieved by collecting data sets relating to individuals having known biological characteristics and analyzing the data sets to identify biomarkers potentially relating to selected biological state classes. Collection of the data set is achieved by the creation (or collection of previously created) of mass spectrometry spectra having perceived particular relevance. Thereafter, the data set is preprocessed through mass alignment, normalization, smoothing and peak identification. The step of classifying is preferably performed through application of a Random Forest algorithm that allows for optimization of the classifiers sensitivity and specificity.

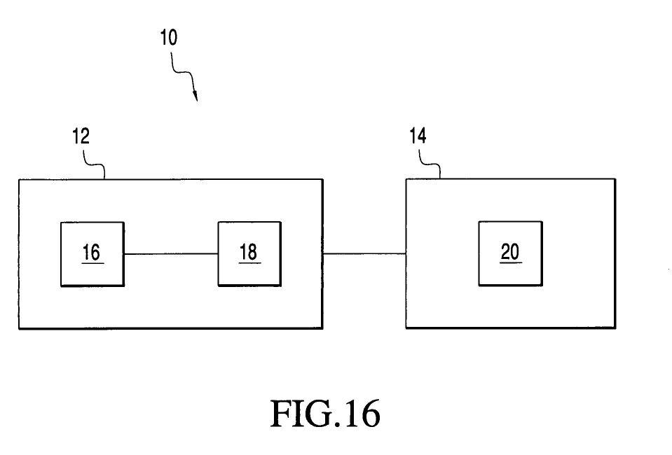

With reference to FIG. 16, the identification system 10 employed in accordance with the present invention may be highly automated and generally includes a mechanism for collecting data sets 12 relating to individuals having known biological characteristics, for example, ovarian cancer, and an analyzing (or classifying) assembly 14 for analyzing data sets to identify biomarkers potentially relating to selected biological state classes. As will be discussed below in greater detail, a variety of automated systems known to those skilled in the art may be employed in the practice of the present invention.

The mechanism for collecting 12 includes means for creating a data set of mass spectrometry spectra 16 and means for preprocessing of the data set 18. Preprocessing includes mass alignment, normalization, smoothing and peak identification.

In accordance with a preferred embodiment, the analyzing assembly 14 includes means for classifying through application of a Random Forest algorithm 20. The analyzing assembly also includes means for defining sensitivity and defining specificity.

More particularly, the present invention provides a comprehensive statistical, computational, and visualization approach to identifying the m/z values for naturally occurring forms of peptide and protein disease biomarkers from raw data collected from mass spectrometric instruments. Although the methodology has been developed based on MALDI-MS spectra, a similar methodology could also be used to analyze electrospray ionization (ESI) mass spectra. The latter might be produced by nanospray or liquid chromatography/MS approaches. Similarly, the methodology that is described would also be suitable for analyzing spectra obtained from state-of-the-art instrumentation such as MALDI and/or ESI equipped Fourier Transform Ion Cyclotron Resonance (FTICR) mass spectrometers.

Mass spectrometric measurements are carried out in the gas phase on ionized samples. There are three basic components in all mass spectrometers. First an ion source ionizes the molecule of interest, e.g. peptides/proteins, then a mass analyzer differentiates the ions according to their mass-to-charge ratio and finally, a detector measures the abundance of ions. Sample ionization is the process of placing charges on neutral molecules. Among ionization methods, electrospray ionization (ESI) and MALDI are the two most commonly used techniques to volatize and ionize the proteins or peptides. ESI ionizes the samples out of a solution and MALDI sublimates and ionizes the samples out of a dry, crystalline matrix via laser pulses.

A mass analyzer is used to separate ions within a selected range of mass-to-charge ratios. Ions are typically separated by magnetic fields, electric fields, or by the time it takes an ion to travel a fixed distance. There are four basic types of mass analyzer currently used in proteomics research: ion trap, time-of-flight (TOF), quadrupole, and Fourier transform ion cyclotron (FT-MS) analyzers. Among them, the TOF mass analyzer is one of the simplest and is commonly used with MALDI. It is based on accelerating a set of ions to a detector with each ion having the same amount of energy. Because the ions have the same energy, yet different masses, they reach the detector at different times. Smaller ions reach the detector first because of their greater velocity and larger ions take longer time, thus the analyzer is called TOF and the mass is determined by the time required for each ion to travel from the source to the detector.

The ion detector allows a mass spectrometer to generate a signal current from incident ions by generating secondary electrons, which are further amplified. Alternatively, some detectors operate by inducing a current generated by a moving charge. Electron multipliers and scintillation counters are the most commonly used and they convert the kinetic energy of incident ions into a cascade of secondary electrons.

The relationship that allows the mass/charge (m/z) ratio to be determined for an individual ion is:
E=½(m/z)v² (1.1)

In this equation, E is the energy imparted to the charged ions as a result of the voltage that is applied by the instrument and v is the velocity of the ions down the flight path. Because all of the ions are exposed to the same electric field, all similarly charged ions will have similar energies. Therefore, based on the above equation, ions that have larger mass must have lower velocities and hence will require longer times to reach the detector, thus forming the basis for m/z determination by a mass spectrometer equipped with a TOF detector. A mass spectrum is created by recording electrical currents produced by different ions reaching the detector with different traveling times. The resulting data format is very simple: paired mass-to-charge ratio (m/z) versus intensities.

The present method and system employ many novel steps in data preprocessing and disease biomarker identification. As briefly mentioned above, data preprocessing includes background subtraction, spectrum alignment (registration), peak identification, normalization, and outlier detection. Disease biomarker identification in accordance with the present invention uses a customized Random Forest algorithm as disclosed by L. Breiman. Breiman L., RandomForest, Technical Report, Statistics Dept. UCB (2001). The algorithm is specially designed for the purpose of parallel computing, e.g., on a 128 node IBM Beowulf cluster. The latter feature is critical for expansion of the dynamic range of the analyses by obtaining and analyzing multiple spectra/sample. The latter might be produced by LC/MS that is carried out either “off-line” or via a liquid chromatograph that is directly coupled to an ESI source of a mass spectrometer. Although a preferred embodiment is disclosed in accordance with the present disclosure, other algorithms are contemplated for searching for features showing distinct patterns among different classes (that is, those samples exhibiting specific biological characteristics) of samples. The present method is built on sound statistical principles and integrates efficient and powerful statistical tools to allow researchers to fully utilize information in the data sets for biomarker identification purposes.

In accordance with a preferred embodiment of the present invention, the present method and system is employed in the identification of peptide/protein disease biomarkers in sera from mass spectrometry data. The mass spectrometry data is preferably obtained from a mass spectrometer equipped with a matrix assisted laser desorption ionization MALDI) source and time-of-flight linear and/or reflectron analyzer.

However, those skilled in the art will appreciate the underlying concepts are not limited to this specific application area. For example, the present method and system may be used to analyze multiple spectra per sample obtained from other types of mass spectrometers (for example, mass spectrometers equipped with liquid chromatographs and electrospray ion sources), to carry out comparative proteome profiling (for example, following tryptic digestion of serum), to analyze all other types of biological samples (for example, tissue and cell extracts), and to analyze data from other types of biomolecule profiling (for example, mass spectrometry-based lipid profiling data). In addition, the preprocessing procedures that have been developed can be applied to other types of experiments where curved data are generated, for example, time-course experiments in microarray studies. As such, it is contemplated that the biomarker identification algorithm of the present invention can be applied to extract useful features from virtually any type of data sets which have a large number of features. In addition, the integrated system can be easily modified for other biomedical applications.

The present method and system has been shown to outperform other existing methods. The present method and system employs a customized Random Forest algorithm having many unique features ideally suited to data sets generated from a wide range of genomic and proteomic studies, which usually have a very large number of features (attributes) but a relatively small number of samples. The underlying computer code employed in accordance with the present invention has been optimized for use on a parallel, cluster computer which will be essential as this biomarker discovery approach is applied to the analysis of multiple spectra/sample following LC fractionation. In this regard, the Random Forest approach has been found to be ideally suited for use on cluster computers which will provide the compute power needed to analyze tens of individual spectra from hundreds of samples in a reasonable time frame.

The present method and system also provides a simple methodology that allows application of proteome analysis to be used on a far wider range of mass spectrometric instrumentation than just a SELDI mass spectrometer. The present method and system refines statistical methods to address a range of important issues including background subtraction, peak identification, and normalization of spectra. The present method and system also introduces visualization tools and a new algorithmic approach to uncovering peptide and protein biomarkers of disease. Using previously published and newly acquired data on sera from control versus ovarian cancer patients, the present disclosure provides practical guidelines for using the underlying concepts of the present invention and suggests how they might be applied in the future to the far more daunting challenge of proteome profiling.

The experimental procedures employed in accordance with the present invention are outlined below. With regard to the collection of mass spectrometry data, and in accordance with a preferred embodiment of the present invention, it is collected in the following manner:

Automated C-18 ZIPTIP Desalting and Spotting onto MALDI-MS Target Plates of Serum and Other Biological Fluids on a PACKARD MASSPREP sample handler. After aliquoting 10 μl of each sample into a 96 well plate, each is acidified by the addition of 5 μl 0.1% TFA. The robot then picks up the first set of 4 C-18 ZIPTIPS (Waters Corporation), which are laboratory pipette tips, and washes them with 50% acetonitrile, 0.1% TFA (trifluoroacetic acid); followed by 0.1% TFA After repeatedly (8×) pulling each sample up into a C18 ZIPTIP and expelling it back into the original sample well, the C18 ZIPTIP is washed 5× with 20 μl 0.1% TFA Bound peptides/proteins are eluted from the C18 ZIPTIP with 10 μl of 50% acetonitrile, 0.1% formic acid into a new 96 well plate. A 2 μl aliquot of each sample eluent is removed, mixed with 0.5 μl alpha-cyano-4-hydroxycinnainic acid matrix in 50% acetonitrile, 0.05% TFA containing an internal standard of 25 fmol bradykinin (M+H C¹² mono-isotopic mass: 1060.569), and then subjected to automated MALDI-MS on a Micromass M@LDI-R or M@ALDI-L/R mass spectrometer.

Automated MALDI-MS Data Acquisition.

The M@LDI-L/R mass spectrometer automatically acquires data in positive ion detection over a mass range currently set at 800-3,500 Da using its reflectron analyzer and 3,450 to 28,000 Da using its linear analyzer. Although the mass range is adjustable, it is difficult to acquire meaningful data below about 800 Da due to interference from the matrix and with a reflectron analyzer, the ionization response drops off substantially as the mass range is increased above about 3,500. Hence, by also analyzing the sample in linear mode, the mass range maybe extended to 28,000 Da (with alpha-cyano-4-hydroxy cinnamic acid matrix). Following acquisition of the reflectron and linear spectra they are joined together to form a continuous spectrum spanning from 800 to 28,000 Da. The mass of 28,000 Da is the upper mass limit for the alpha-cyano-4-hydroxy cinnamic acid matrix. This mass range could be extended up to >100,000 Da if the sample was re-spotted using a matrix suitable for large MW proteins, such as sinapinic acid.

Currently, the M@LDI-L/R sums 10 individual laser shots into one spectra with the laser operating at 10 Hz. The laser moves in a random walk around the target well, acquiring data from a maximum of 20 different locations within each 2 mm diameter well. A spectra is considered “acceptable” if it has a signal that is >2% above background noise, less than 95% of saturation, and in the case of the reflectron spectrum, if there is at least one m/z detected between 1,125 Da and 3,500 Da. The M@LDI-L/R is programmed to retain up to 40 acceptable spectra, but if it sequentially acquires 4 unacceptable spectra, it will move to another location within the same target well. The instrument uses an incrementally increasing laser percentage to heat up the target spot to acquire acceptable spectra, while still having the lowest possible laser energy, which provides the best possible mass resolution. If the M@LDI-L/R acquires 20 acceptable spectra at one position, it will then move to another position in the same sample well, and will acquire another 20 acceptable spectra, unless interrupted by 4 unacceptable spectra. Once the M@LDI-L/R has shot (not acquired) 40 acceptable spectra, it will move to the next sample well. This means there can be a maximum of 40 acceptable spectra acquired for each sample, and that if at no point it acquires acceptable data, it will try up to 10 different locations within the same sample target well before moving on to the next sample. Typically, the resulting spectrum represents the average of 20-40 spectra. The expected mass resolution is 14,000 at M+H 2,465 and mass accuracy is better than ±70 ppm. Each (averaged reflectron and linear) MALDI-MS spectrum is converted to a text file listing of 91,400 m/z versus intensity data points spanning the m/z range from 800-3500 Da and nearly 40,000 data points spanning from 3500 Da to 28,000 Da which is then suitable for further analysis.

Additional information on both automated desalting of serum samples and MALDI-MS data acquisition can be found in Appendix A, which is attached hereto

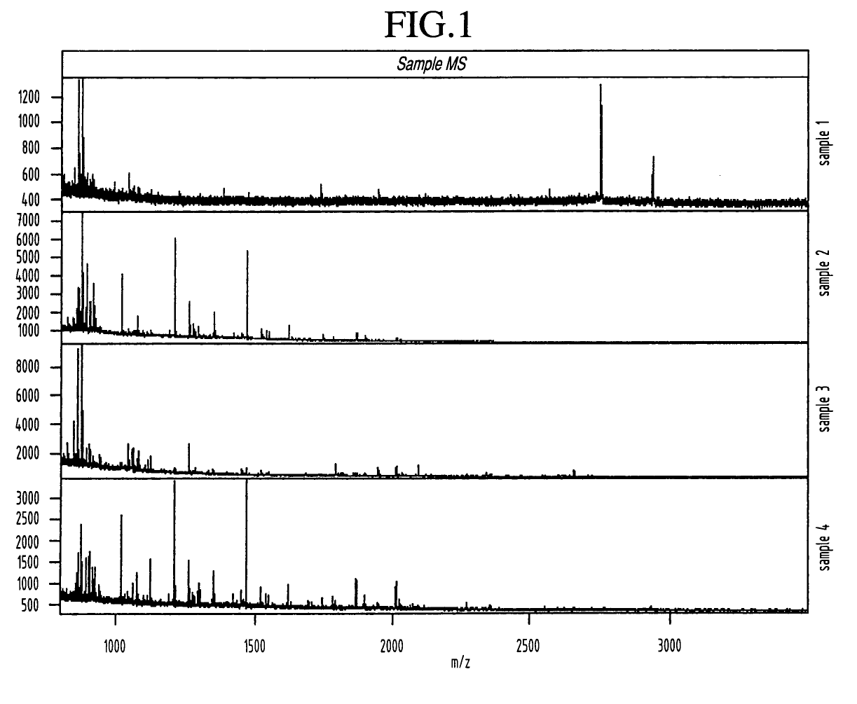

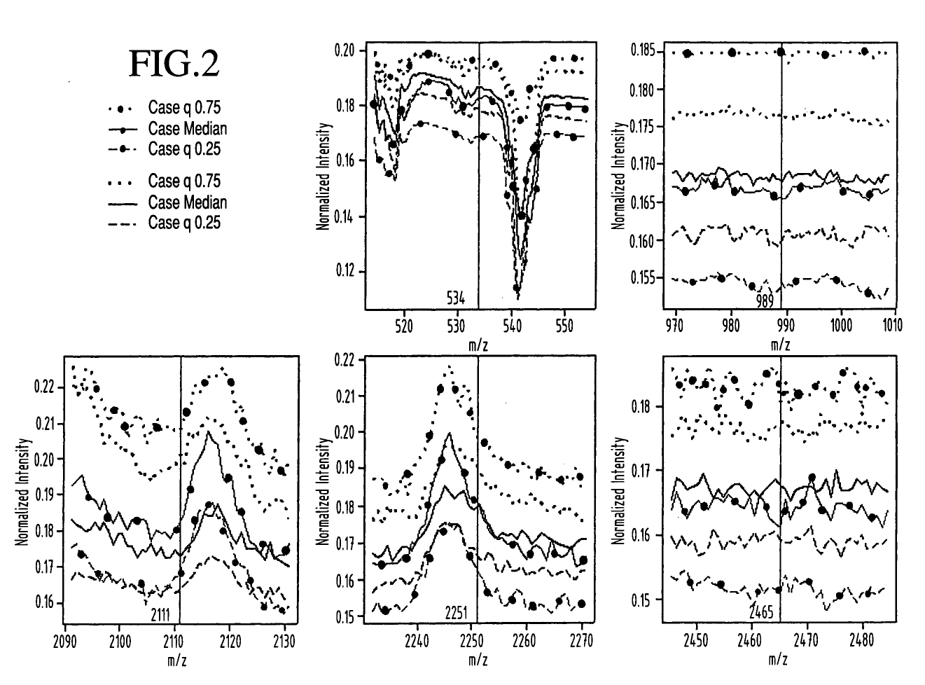

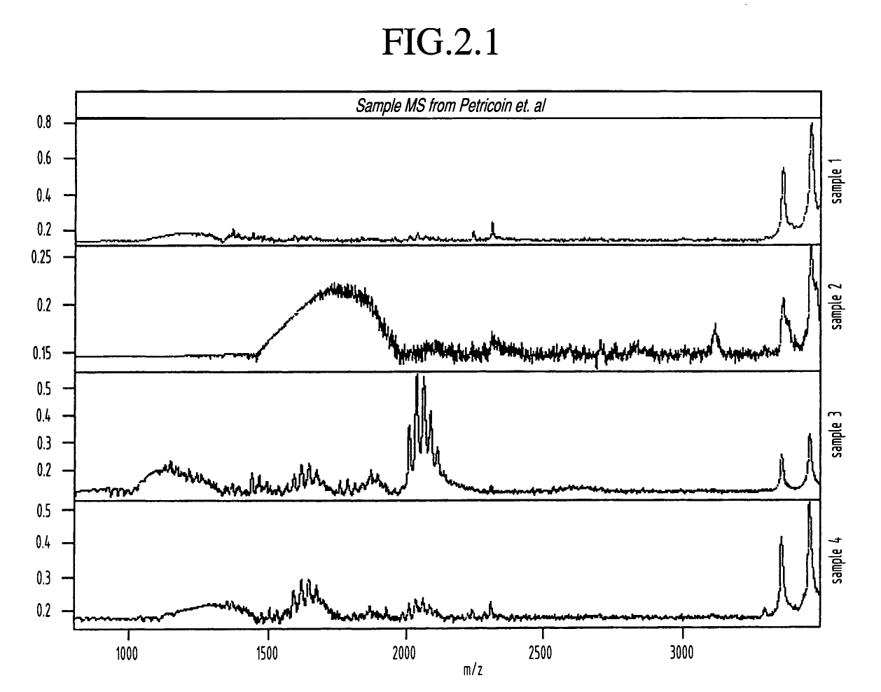

The data that results from MALDI-MS analysis has a very simple format consisting entirely of paired intensity versus mass/charge data points. Because MALDI-MS of peptides primarily produces singly charged species, the mass/charge ratio is usually equal to the mass. FIG. 1 shows raw MALDI-MS spectra acquired as described above on four serum samples from ovarian cancer patients in the National Ovarian Cancer Early Detection Program clinic at Northwestern University. Perhaps the most apparent feature of these spectra is their diversity both with respect to the peptides that are present in each and their relative MALDI-MS response, which is indicated also by the variations in the intensity scales on the y-axis. This high level of diversity suggests that reasonably large numbers of samples will need to be analyzed to find commonalities that might be used to differentiate serum from ovarian cancer versus normal patients and that individual biomarkers are likely to have modest predictive value.

A less apparent challenge presented by the data in FIG. 1 is that each reflectron spectrum is composed of 91,400 individual data points. This means that if the entire spectrum is used in the search for biomarkers, there will be a very large ratio of data points/samples. This presents unique challenges as will be described in more detail below.

Statistical issues in the analysis of mass spectrometry data can be broadly classified into three categories: preprocessing, peak identification, and biomarker identification. Data visualization is an important element in biomarker identification. Data preprocessing includes mass alignment, normalization, background subtraction, smoothing and peak identification. Appropriate normalization methods are needed to ensure that all samples contribute reasonably equally to the analysis.

Background subtraction removes noise, which actually accounts for most data points.

Moreover, the observed mass spectrometry intensity has a wide dynamic range (0 to 20,000 in the case of reflectron spectra). This further challenges statistical analysis of mass spectrometry data. Peak identification is important so that biomarker identification is focused on those regions of the spectra that result from ionization of peptides as opposed, for instance, to differences in baselines. Since each peptide that ionizes produces several data points/peak and with a reflectron analyzer, multiple isotope peaks, it is important that only one (that is, the best in terms of discriminating control from experimental samples) m/z versus intensity data point be chosen for each peptide biomarker.

Statistical approaches designed to analyze data sets that contain a much smaller number of features compared to the 91,400 m/z versus intensity data points that compose each of the spectra in FIG. 1, cannot be applied to mass spectrometry-based biomarker discovery due to challenges that arise from the large data point/sample number ratio. Instead, the present method and system employ techniques that are not compromised by this feature which is inherent to mass spectrometry data sets. Although statistical methods are essential for preprocessing mass spectrometry spectra and for identifying biomarkers that can best discriminate large numbers of control from experimental samples, it is equally important that visualization tools be developed that can effectively identify possible anomalies in the data set and provide a final confirmation that the selected biomarkers appear to be reasonable and to derive from peptide ionization.

As discussed above, preprocessing of mass spectrometry data aids in the effectiveness of the present invention. In accordance with a preferred embodiment of the present invention, prior to identifying peaks and initiating the search for potential biomarkers, each raw MS data set is subjected to four sequential procedures (mass alignment, logarithmic transformation, background subtraction, and normalization) that are designed to optimize it for biomarkers based on a customized Random Forest algorithm as will be summarized below in detail.

Mass alignment. In an ideal experiment, all ions will have the same kinetic energy E and will travel through the exact same drift region length. However, some initial kinetic energy distribution will be present in the ion population and there will be slight spatial variations in the travel length from the target plate which will produce corresponding variations in the traveling time and thus the measured m/z ratio for ions with exactly the same mass. This problem is partially solved by using time delayed ion extraction (Randy M. Whittal and Liang Li, High-Resolution Matrix-Assisted Laser Desorption/Ionization in a Linear Time-of-Flight Mass Spectrometer, Anal. Chem 67, 1950-54 (1995); Robert S. Brown and John J. Lennon, Mass Resolution Improvement by Incorporation of Pulsed Ion Extraction in a Matrix-Assisted Laser Desorption/Ionization Linear Time-of-Flight Mass Spectrometer, Anal. Chem 67,1998-2003 (1995)) in MALDI-TOF, but as a side effect it also changes the linear relationship between m/z and t² (i.e., v²=D²/t² where D is the distance traveled) in equation (1.1). A first order approximation can be used:
m/z=a+bt², (1.2)
where a and b are constants for a given set of instrument conditions and are determined experimentally from flight times of ions of at least two known masses (calibrants). In practice, higher order approximations have been proposed to achieve higher accuracy. Johan Gobom, Martin Mueller, Volker Egelhofer, Dorothea Theiss, Hans Lehrach, and Eckhard Nordhoff, A Calibration Method That Simplifies and Improves Accurate Determination of Peptide Molecular Masses by MALDI-TOF MS, Anal. Chem. 74,3915-3923 (2202). Even with the use of internal calibration the maximum observed intensity for an internal calibrant may not occur at exactly the same corresponding m/z value in all spectra. For this reason, spectra can be further aligned based on the maximum observed intensity of the internal calibrant, after which there are still some problems with local peak shifting. Useful statistical methods need to be developed to address this problem.

Although spectra obtained from the M@LDI-L/R instrument used in this study were internally calibrated by adding bradykinin to all samples, slight variations (that is, within the expected mass accuracy of <70 ppm) were seen in mass values for the same relative data points in different spectra. To circumvent this challenge, data points are numbered consecutively by assigning the observed mass measurement value that is closest to the expected MH+for the C¹² isotope of bradykinin, which is 1060.569, as data point zero.

Logarithmic transformation. Measured protein/peptide concentrations in samples like human serum have a vast dynamic range (more than 10¹⁰-fold) that spans from 35-50 mg/ml for serum albumin down to at least 0-5 pg/ml for interleukin 6. Anderson, N H and Anderson, N G, The human plasma proteome, Mol. & Cell. Proteomics 1, 845-867 (2002). Although mass aligned spectra of serum and other biological samples can be directly analyzed, the relatively large variations in the measured intensities are likely to make most statistical procedures unstable, thus making it more difficult to extract information from the MS dataset. In addition, the large magnitude of the intensities will make most numerical programs unstable.

Although mass aligned spectra can be directly analyzed, the relatively large variations in the measured intensities are likely to make most statistical procedures unstable, thus making it more difficult to extract information from the mass spectrometry data set. In addition, the large magnitude of the intensities will make most numerical programs unstable. As a straightforward approach to minimize these challenges, we take the logarithms of the intensities to reduce the variation of the raw dataset. Therefore, the numerical variations in the intensities across the spectrum and all the samples are substantially reduced.

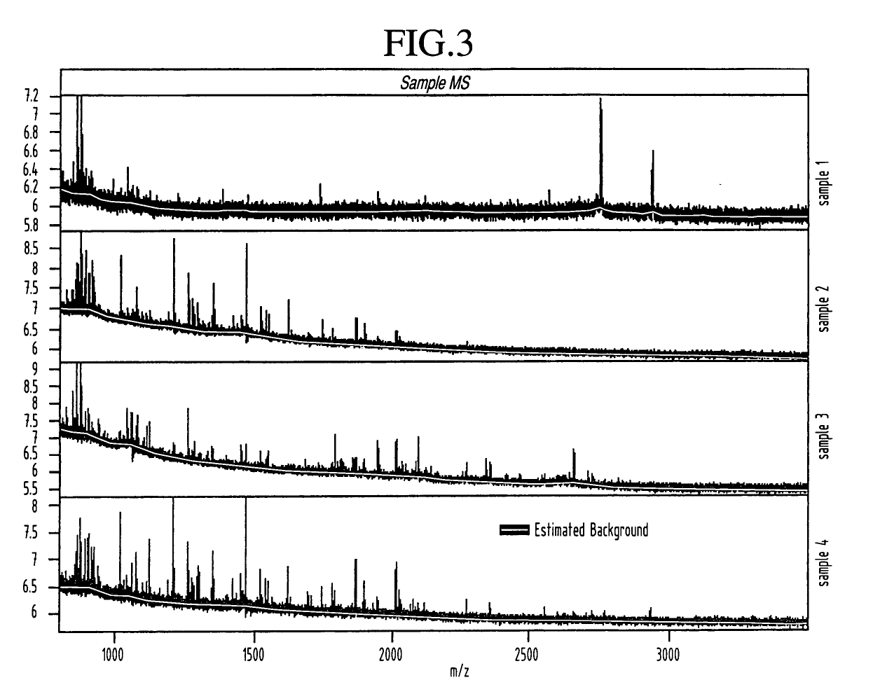

Background subtraction. Chemical and electronic noise produce a background intensity that typically decreases with increasing m/z values and that is present regardless of whether or not a sample has been deposited onto the target. To minimize the impact of noise and the overall downward sloping baseline trend, we estimate the background intensity level by assuming that nearby mass spectrometry points share common background information. This is achieved by using the Robust locally Weighted Regression and Smoothing Scatterplots (also known as ‘lowess’) method to estimate local background levels by performing a robust linear regression using a sliding window across each spectrum Cleveland, W. S. Lowess: A program for smoothing scatterplots by robust locally weighted regression; The American Statistician 35, 1981, 54. Although one skilled in the art could carry out such a procedure, it must be optimized for MS data by choosing the proper size window. Other approaches such as quantile regression and wavelet transformations are also being explored for their relative usefulness in estimating background levels and removing noise from MS data. FIG. 3 illustrates the result of this background estimation method using lowess for several samples.

Smoothing. High frequency noise is one contribution to the background that is apparent in MALDI-MS spectra. Smoothing functions can also be used to reduce high-frequency noise, thus minimizing noise spikes and aiding interpretation.

Normalization. To obviate differences in the overall level of intensities that are recorded for a given sample and that might result from experimental variables such as pipetting or uneven sample deposition/matrix crystallization on the target, each spectrum is linearly normalized to try to ensure that all samples contribute as equally as possible to the search for biomarkers. Since each data point in each spectrum is normalized with the same factor, this procedure does not change the observed peak-to-peak ratios in a spectrum; that is, both the raw and normalized spectra will have exactly the same overall m/z versus intensity profile. Normalization is accomplished by assuming there are n samples: (X1, X2, . . . , Xn), each having 100,000 intensities, and that we would like to find n normalization factors: (f1, f2, . . . , fn) to make (X1/f1, X2/f2, . . . , Xn/fn) as comparable to each other as possible. Those skilled in the art will readily appreciate the complete normalization process. To estimate each fn factor we first calculate for each data point the overall median intensity, which is noted as Xm, for that m/z value across all samples. For each spectrum we then fit the ordinary least square regression of Xm˜Xj without intercept, denote the regression coefficient by cj, and we use fj=cj as the normalization factor for each of the data points that together make up that sample's spectrum. We exclude those samples with cj>2 or cj<1/2 for further analysis.

Although several normalization approaches are possible, one straightforward approach is to determine a linear normalization factor that will minimize the summed difference between all observed intensities in an individual spectrum and the calculated median spectra for all of the samples. However, the validity of such approaches needs to be rigorously investigated.

Once the raw mass spectrometry data is preprocessed as described above, the spectra are analyzed for peak identification. Intensity measurements from current mass spectrometry technology tend to be quite noisy with approximately 80% of the data points in spectra like those in FIG. 1 deriving from both electrical and chemical noise. Therefore, noise filtering is a necessary and indispensable step to allow biomarker identification to be concentrated on those data points that derive from peptide/protein ionization and that might represent useful biomarkers. Although the following procedure has been adopted in accordance with the currently preferred embodiment of the present invention for peak identification, other methods for peak identification and alignment are contemplated for use in accordance with the spirit of the present invention. In the present embodiment, the following three criteria are used to define peaks

Noise Filtering. In accordance with a preferred embodiment of the present invention, we take advantage of our finding that approximately 80% of MALDI-MS data points acquired on serum samples result from noise and set a minimum intensity level that can serve as an effective and simple global noise filter. Hence, the assumption is made that only the top 20% of the observed intensities of each linearly normalized spectrum are likely to contain useful biomarkers (that is, only the top 20% of the observed intensities are likely to result from ionization of peptides).

We note that the 20% value is only an example. In practice, this parameter can be adjusted based on the quality of the spectra. That is, this represents a global criterion that be easily adjusted for different data sets and easily confirmed as being reasonable by plotting the top 20% of intensities for some of the higher intensity spectra obtained and confirming that no significant peaks have been filtered out as noise. Alternative approaches might rely on criteria based on local measures and treating different regions of the mass range differently. High-frequency noise filtering also may improve upon this global criterion.

Peak Test. The assumption is made that only data points in completely or partially resolved peaks (that is, data points in partially resolved peaks may represent the intensity sum of a useful biomarker superimposed on an unrelated, non-biomarker peptide ion) result from peptide ions and are likely to be useful. To pass this test, at least 3 out of 4 successive data point intensities before or after each candidate biomarker data point must show a progressive increase or decrease in background corrected, normalized peak intensity. The basic concept is to search for local maximum and that by putting some constraints on the data it is also possible to filter out some noise spikes. Additional work is being carried out to further improve the peak detection methodology. A few plots of high and low intensity spectra that are made before and after imposition of the peak test serve as a quick visual confirmation of the suggested stringency, which can be easily altered as needed for different types of data sets. To further narrow our focus to peaks that are found in a reasonable fraction of samples, we require that at least 10% of the cases or controls need to pass the peak test for any peak to be considered a useful biomarker. While the value of 10% constraint appears to work well for the serum samples used in the present study, this parameter may need to be adjusted for different data sets (e.g. for cell extracts and for data acquired with other MS sources).

Unique Peptide Ion Test. Following peak identification, it is important that multiple biomarkers that arise from the same peptide are eliminated as there is no benefit in having multiple biomarkers that all originate from different isotopes of the same peptide ion. To accomplish this objective we require that all potential biomarkers must have m/z values that differ from each other by at least 3.1. This criterion will thus eliminate multiple biomarkers that all derive from the monoisotopic [C¹²] and the first two higher isotopic peaks (containing, for instance, one and two C¹³ atoms respectively) in an envelope that derives from the same peptide. Since it is quite possible (for example, if there are incompletely resolved, unrelated peptide ions that overlap with the C¹² isotope peak of a biomarker peptide ion) that the “best” isotopic representative of a biomarker ion is not the C¹² isotope, we would not want to limit our search to only the monoisotopic ion. Given the potential for overlapping peptide ions, we also would not want to merge the isotope peaks and represent the biomarker as the sum of the component contributions of its individual isotopes. Rather, when multiple biomarkers are found that arise from a common peptide ion, we need to define statistical criteria for selecting the best biomarker for that peptide.

Our current strategy is to rank all biomarkers that appear to derive from the same peptide based on their ability to differentiate cases from controls and to then select the best one. In accordance with a preferred embodiment of the present invention, the rank is based on F-statistics for testing differences. However, those skilled in the art will certainly appreciate the other test statistics that could also be used for this purpose without departing from the spirit of the present invention.

Once the data sets are collected and processed, biomarker identification may then take place. As discussed above, and in accordance with a preferred embodiment of the present invention, a customized Random Forest program is used as a classifier in biomarker identification. The Random Forest algorithm in accordance with the present invention is used to identify approximately 20-40 biomarkers whose intensities can best discriminate all cases from control samples in a training set. As will be best appreciated from the following disclosure, biomarker selection is ultimately optimized by increasing the training set size until the ability of the resulting biomarkers to classify one or more testing sets is maximized. If the resulting classification error is too high, the next logical step would be to fractionate the sample (e.g., by liquid chromatography and utilize a similar strategy to optimize the number of fractions that should be analyzed by MALDI-MS for each sample.

This customized Random Forest program employs appealing features in that it combines bagging with random feature selection. Bagging results in pooling multiple classifiers from perturbed versions of the original dataset to increase predictive accuracy. For our data set, the number of m/z versus intensity variables is large compared to the number of samples, so it is not surprising that each individual variable has small predictive power. Under these conditions it is unwise to just select a single or even a few “best” variables for classification. Using the random feature selection will increase our predictive accuracy. A side product of bagging is out-of-bag prediction for each sample, which provides a very accurate estimate of the relative importance of each variable (that is, biomarker) that is similar to cross-validation. Breiman, L. Random forests. Machine Learning 45, 1(2001), 5-32.

Enhanced accuracy of the classifier may be achieved by setting minimum importance values criteria for use of each biomarker, thus ultimately improving predictive ability. In addition, a minimum confidence level for classified samples may also be set in an effort to further improve the results. Those samples not meeting the minimum confidence level could then be re-analyzed multiple times with the resulting spectra being averaged which might then allow them to meet the minimum confidence level.

In particular, and in accordance with a preferred embodiment of the present invention, a Random Forest algorithm as disclosed by Breiman is utilized. Breiman, L. Random forests. Machine Learning 45, 1 (2001), 5-32. Random forest combines two powerful ideas in machine learning techniques: bagging and random feature selection. Bagging stands for bootstrap aggregating, which uses resampling to produce pseudo-replicates to improve predictive accuracy. By using random feature selections, we can significantly improve our predictive accuracy. It works as follows:
(1) Sample with replacement to form N bootstrap samples {B₁ . . . B_{N}}.
(2) Use each sample Bₜ to construct a Tree classifier Tₖ to predict those samples that are not in Bₜ (called out-of-bag samples). These predictions are called out-of-bag estimators.
(3) Before using Tₖ to predict out-of-bag samples, if we randomly permute the value for one variable for these out-of-bag samples, intuitively the prediction error is going to increase and the amount of increase will reflect the importance of this variable.
(4) When constructing Tₖ, at each node splitting we first randomly select m variables, then we choose one best split from these m variables.
(5) Final prediction is the average of out-of-bag estimators over all Bootstrap samples.

Currently we are exploring the use of weighted sampling at each split so that more informative features maybe sampled. This approach is highly compute intensive and requires the use of parallel computing.

The present method and system provides an effective visualization method appropriate for comparing large numbers of complex mass spectrometry datasets and the regions around selected biomarkers. In accordance with the application of the present method, it is believed that a plot can reveal critical underlying features of the dataset that might otherwise be missed and a plot also can serve as a visual control for a complex statistical analysis. Obviously, if one of the best biomarkers selected by an algorithm is not “visible” on an overall median difference plot comparing all case to all control samples, then it might be appropriate to further examine why this particular m/z versus intensity data point was selected by the algorithm as a biomarker. In the ovarian cancer biomarker analysis that follows, several types of plots will be shown that provide effective visualization of MALDI-MS datasets.

Reproducibility of MALDI-MS Spectra

There are several steps in the overall procedure outlined in accordance with the present method that would be expected to have a certain level of variability that would manifest in the resulting mass spectrometry spectra as overall differences in intensity and/or differences in relative intensities of individual peaks. These steps include the robotic liquid handling, C-18 ZIPTIP desalting, spotting onto the MALDI target, and the actual data acquisition itself. We have examined the reproducibility of the last step by analyzing individual spectra obtained from the same spotted MALDI-MS target and we have examined the robotic processing steps by comparing summed MALDI-MS spectra acquired on aliquots of the same sample that have been individually desalted manually and/or spotted by the MassPrep robot.

As will be discussed below in greater detail, the present method and system provides enhanced reproducibility improving efficacy. In particular, the present method and system provides for reproducibility of the whole process including ZIPTIP/spotting/data acquisition, reproducibility of spotting/data acquisition and reproducibility of individual spectra acquired on a sample and that are summed together to give the output.

It is further contemplated that the present method and system may be employed with the introduction of 10% intensity peak expansion of the training set from 24 to 48 etc., graphs of the impact of increasing the training set size and the number of biomarkers on the success rate at classifying 2×24 testing sets. The latter is perhaps the most important element as the graph of the size of the training set as a function of the success rate at classifying two known test sets (each of which contain approximately equal numbers of control and disease samples) provides a very facile means to determine how large the training set needs to be to obtain biomarkers that can optimally classify test samples. Once the training set size has been optimized (at the lowest number of samples that provides biomarkers with the highest success rate at classifying the “unknown” test set), then the number of biomarkers included can then be similarly optimized.

To increase the probability of detecting more peptides and to improve the accuracy of the intensity measurements, Micromass' M@LDI™ systems automatically acquire up to 40 individual spectra on each target with the final reported intensity being the sum of these individual spectra. Each individual spectrum in turn is the summed ion intensity detected from 10 laser shots at a given position on the target. As a result of variation in automated sample aliquoting and desalting, deposition on the target, matrix crystallization, and ion detection; the overall intensity measurements between two different aliquots of the same sample often vary by at least 4-fold. To assess the extent of this variability that may result from acquiring multiple spectra from the same target, we examined the variability among the 40 individual spectra acquired from one target that had been robotically spotted with a serum sample from a control patient. Each reflectron spectrum contains 91,268 m/z versus intensity data points that cover the range extending from 800 Da-3500 Da. Based on the minimum intensity level test (that is, noise filtering) and the peak test for the summed intensities, 130 peaks were selected for analysis. For every peak there are 40 intensity measurements from 40 spectra, thus we calculated the coefficient of variation and standard deviation for these 40 measurements before/after log-transformation. Hence, there are 130 standard deviation and coefficient of variations for these 130 peaks.

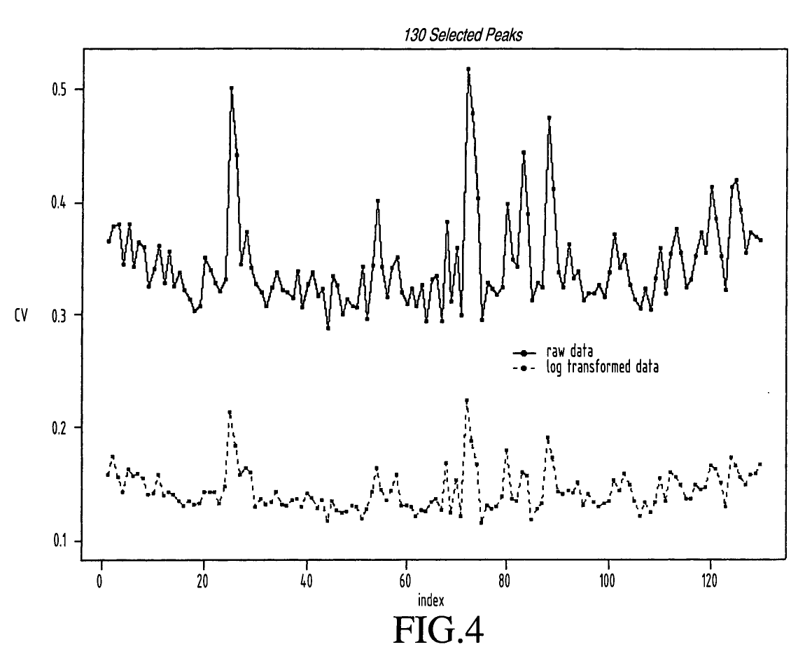

Basically, we want the standard deviation to be small so the intensity measured for each peak will be as accurate as possible. Standard deviation and mean are unit dependent while the coefficient of variation is independent of the units of measurement. We use the relative variation, i.e., coefficient of variation, to measure the variation in the measurements taken for each peak with a smaller coefficient of variation resulting in a more accurate measurement. We can see from FIG. 4 that taking log of the intensities significantly reduces the variation as measure by the coefficient of variation.

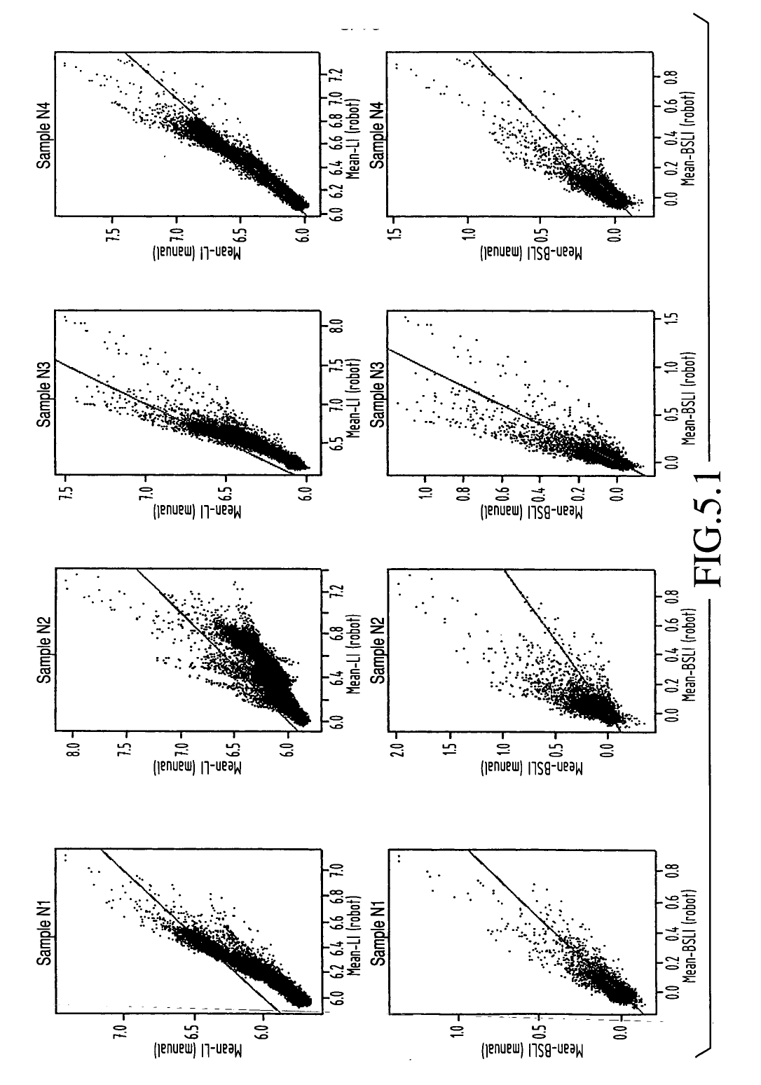

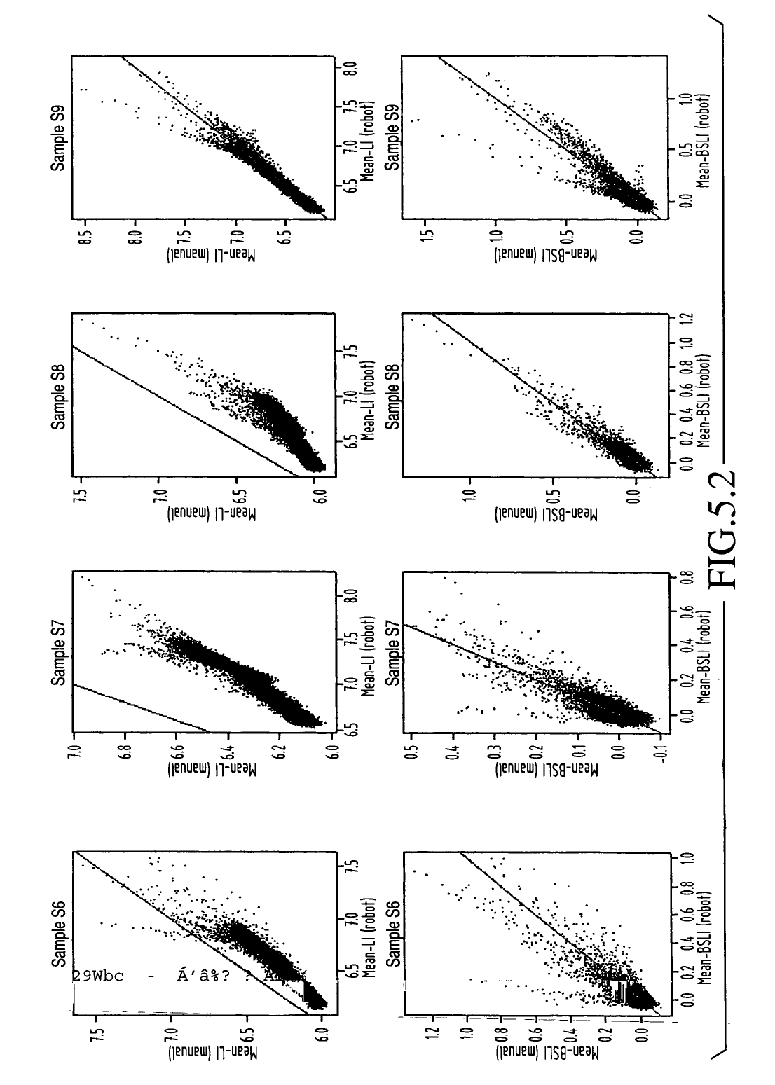

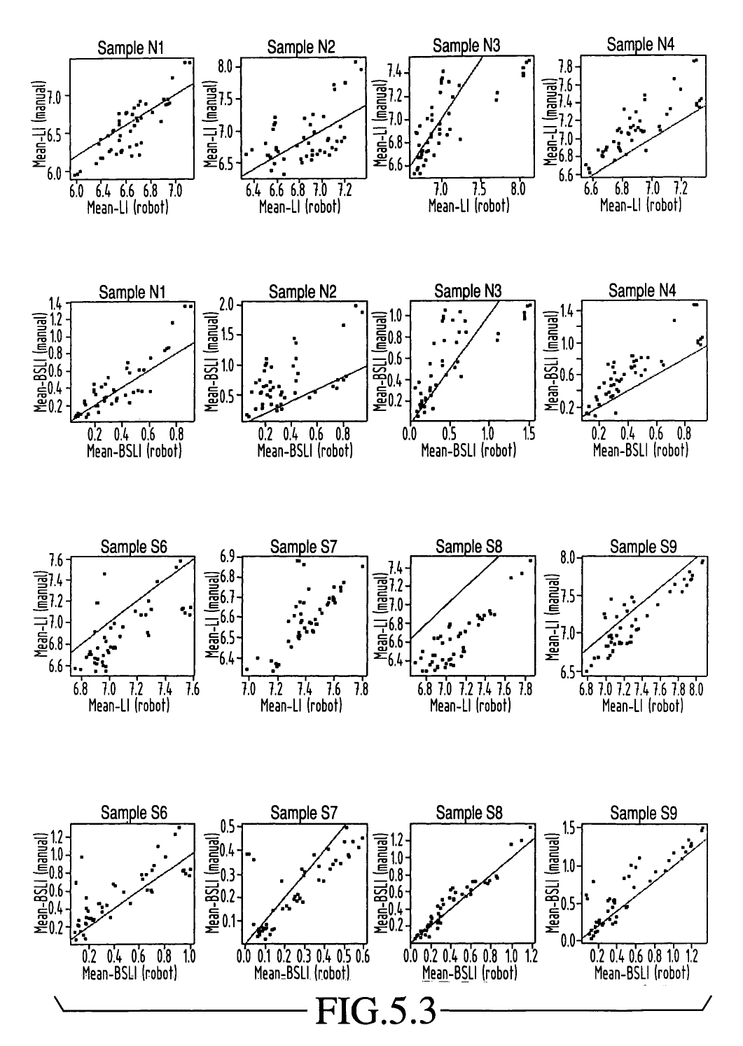

We have examined data from 4 robotically and 2 manually processed and spotted aliquots of 7 samples and 4 robotically and 1 manually processed aliquot of another sample. In FIGS. 5.1, 5.2 and 5.3 we plot the mean intensities of manually processed samples vs. the mean intensities of robotically processed samples. In the plot we compare the log intensities (LI) and background-subtracted log intensities (BSL1), and we include a best fit diagonal line. We can see that overall they agree well after background subtraction.

For these 47 replicate samples, we further identified 49 peaks. In the following plot, we further compare manual vs. robotic procedures at these 49 points, and we also calculate the coefficient of variation at these 49 peaks for 4 robot measurements.

### EXAMPLE 1

### Biomarker Analysis of Serum Samples from Ovarian Cancer Versus Control Patients

The 95 ovarian cancer and 92 control serum samples used in our analysis were obtained from the National Ovarian Cancer Early Detection Program at Northwestern University Hospital and correspond with some of the same samples that were used previously by Petricoin et al. As described above with reference to the experimental procedures, all samples were desalted via adsorption/elution from C18 ZipTips and were then subjected to MALDI-MS on a Micromass M@LDI-R instrument (note that at the time this data was acquired the Micromass M@LDI-R instrument had not yet been upgraded to the linear/reflectron (L/R) version) with all procedures being highly automated. The detailed protocol can be found in Appendix.

This data set consists of mass spectrometry spectra that were obtained on serum samples from 95 patients with ovarian cancer and 92 normal patients. These spectra extend from 800 to 3500 Da and were acquired with the reflectron analyzer of a Micromass M@LDI-R instrument. Twelve samples had poor spectra and they were excluded from further analysis.

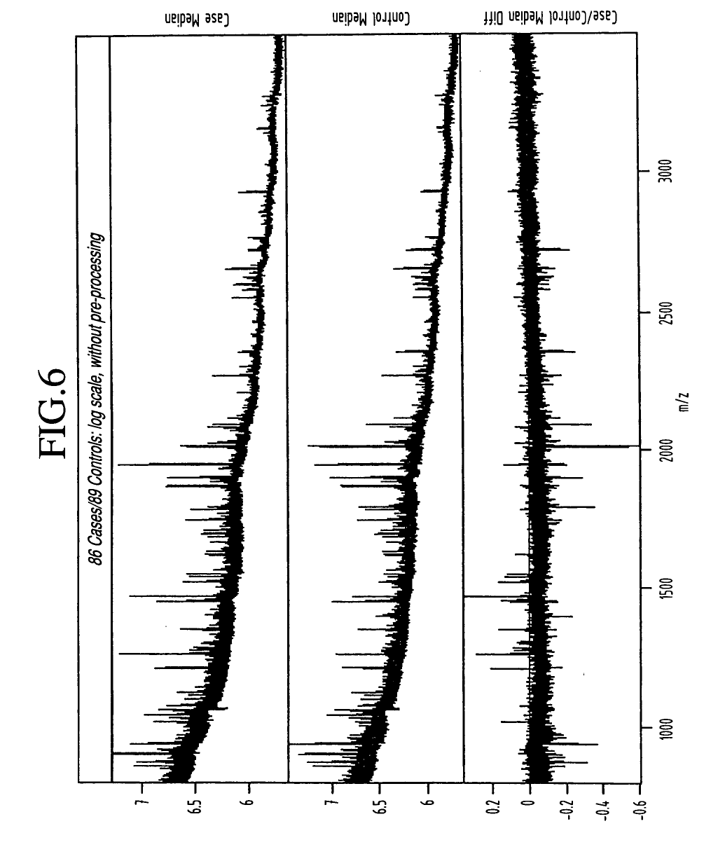

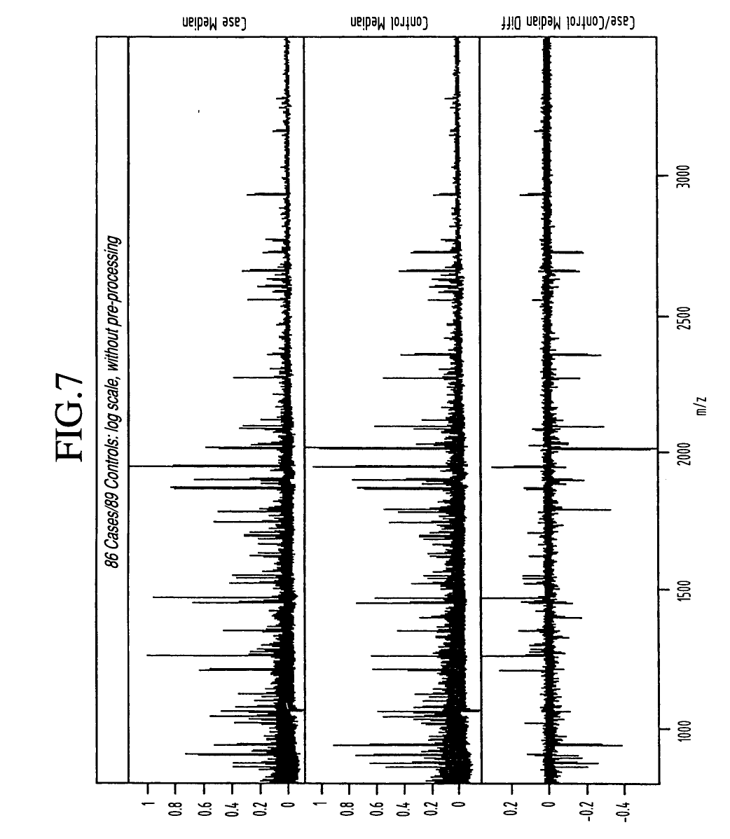

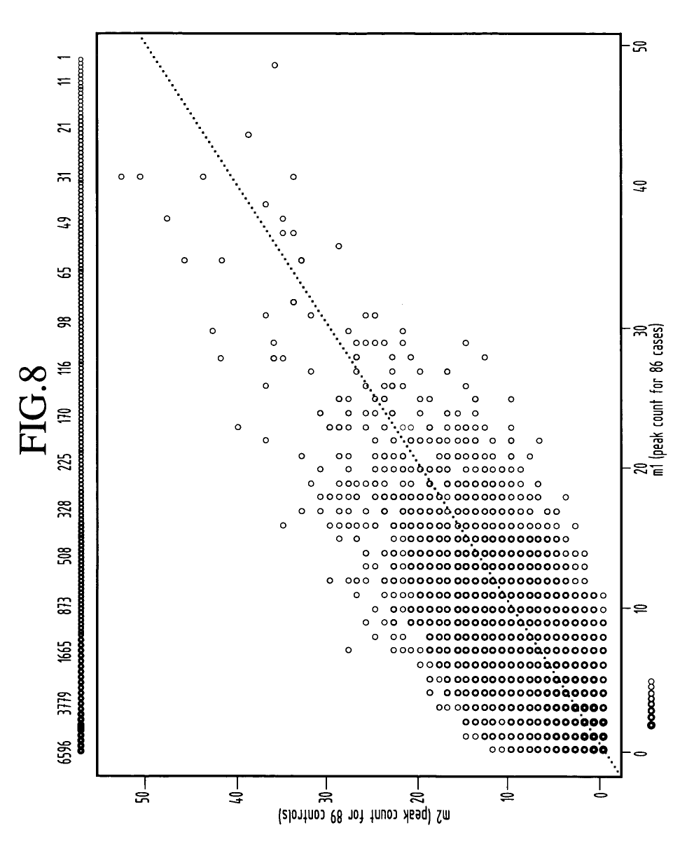

We then preprocessed the raw data sets. Our first step is mass alignment; the resulting dataset has 91254 m/z measurements. FIG. 6 shows the overall case and control median log intensities based on these samples. FIG. 7 shows the median intensity after preprocessing (background subtraction and normalization). For these normalized samples, we apply our peak identification procedure and find the peak distribution for each data point. FIG. 8 shows the distribution of peaks for all samples at each point. It can be seen that the identified peaks are only found in a small proportion of the cases and controls. There is not a single peak that is found in all cases or controls which confirms the need for multiple biomarkers.

For these identified peaks, we calculate the two-sample T-statistics, and rank them based on their absolute values. The top 3500 peaks are used in Random Forest analysis in accordance with the present invention. We can vary the number of peaks used in Random Forest analysis for different datasets. For our dataset, 3500 seems to lead to represent an optimum number.

We applied the Random Forest program to the normalized dataset with selected peaks and have an 8% error rate for 89 cancer samples, a similar 8% error rate for 86 normal samples and thus an overall 8% error rate. The error rate is based on out-of-bag estimation. It is important to point out that these numbers are somewhat misleading in that they are based on internal CV and under-estimate the true error rate. In our later analysis, we have applied CV with feature selection within each training set, and the error rate is higher, about 25%. We expect this error rate will be substantially decreased as we acquire and merge together both reflectron and linear spectra for each sample (thus extending the analysis range up to 28,000 Da) and as we begin to fractionate samples and analyze multiple spectra/sample.

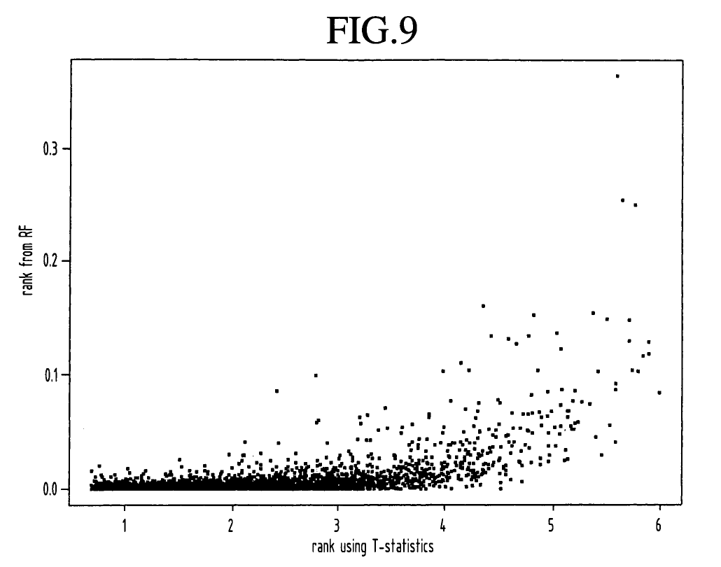

The Random Forest algorithm also produces variable importance measures that reflect the relative importance of each variable for prediction. We can compare these measures for different peaks to the ranks of these peaks based on their T-statistics. FIG. 9 plots the ranking measures of selected peaks based on T-statistics and the importance measures. We can see that while both measures will be able to capture a common set of variables, there do exist discrepancies between these two measures.

### EXAMPLE 2

In accordance with a preferred embodiment, the principles outlined above were applied. In particular, ovarian cancer and control serum samples were obtained from the National Ovarian Cancer Early Detection Program at Northwestern University Hospital. The Keck Laboratory then subjected these samples to automated desalting and MALDI-MS on a Micromass M@LDI-L/R instrument (as opposed to the Micromass M@LDI-R instrument used in Example 1) as described generally in Appendix A.

The M@LDI-L/R mass spectrometer automatically acquires two sets of data in positive ion detection mode. The mass range acquired is dependent on the mass analyzer being used, with 700-3500 Da for reflectron and 3450-28000 Da for linear. This dataset consists of merged mass spectrometry spectra that extend from 700 to 28000 Da and that were obtained on serum samples from 93 patients with ovarian cancer and 77 normal patients.

As mentioned above, Random Forest combines two powerful features: Bootstrap to produce pseudo-replicates and random feature selection to improve prediction accuracy. Breiman, L. Random Forests. Machine Learning 45, 1(2001), 5-32. Random Forest can also estimate the importance of features according to their contribution to the resulting classification. (For a more detailed description of the algorithm see Wu, B., Abbott, T., Fishman, D., McMurray, W., Mor, G., Stone, K., Ward, D., Williams, K., and Zhao, F Comparison of statistical methods for classification of ovarian cancer using mass spectrometry data. Bioinformatics 19, 13 (2003a), 1636-1643, which is included as Appendix B.) From Random Forest program we can get the posterior probability of belonging to each class for each sample. Based on these posterior probabilities we evaluate the sensitivity, specificity and classification errors.

We summarize our mass spectrometry dataset for n samples in a p by n+1 matrix: (mz, X,) (mz, X₁, . . . ,Xₙ) where p is the number of m/z ratios observed, m/z is a column vector denoting the measured m/z ratios, and the xᵢ are the corresponding intensities for the i-th sample. We use vector Y=(yᵢ) to denote the sample cancer status. Our goal is to predict yᵢ based on the intensity profile X′ᵢ=(x₁ᵢ, x₂ᵢ, . . . ,xₚᵢ). Assume that we have g classes. Random Forest classifier partitions the space X of protein intensity profiles into g disjoint subsets, A₁, . . . , A_{g}, such that for a sample with intensity profile X=(x₁, . . . , xₚ.,) E Aj the predicted class is j.

Classifiers are built from observations with known classes, which comprise the learning set (LS) L={(X₁, y₁), . . . , (Xn_{L}, yn_{L})}. Classifiers can then be applied to a test set (TS) T={(X₁, . . . , Xn_{T}}, to predict the class for each observation. If the true classes y are known, they can be compared with the predicted classes to estimate the error rate of the classifiers.

We denote the Random Forest classifier built from a learning set L by C(., L). Given a new sample (X, y), we can represent C(x, L) by a g-element vector (C₁, . . . , C_{g}). If we want a hard-decision classifier, we will have Cₖ=1 and C_{i≠k}=0, that is, it predicts sample (X, y) to belong to class k. Or we can have a probability output, Pr (Cᵢ=1)=Pᵢε[0,1] and Σᵢ₌₁, . . . ,_{g} Pᵢ=1, that is, it predicts the probability that sample (X, y) belongs to class k is Pₖ.

For the ovarian cancer data set considered in accordance with this example we only have two classes, cancer (y=1) and normal (y=2) samples. For two-class classification problems we can define sensitivity (θ) and specificity (η). They are inherently related to classification errors. The relationship between sensitivity and 1—specificity is well known as ROC curve in medical research. Sensitivity is also known as true positive rate, which is the probability of classifying a sample as cancer when it actually derives from a patient who has the cancer, i.e. Pr(C(X, L)=1|y=1). Specificity is also known as the true negative rate, which is the probability of classifying a sample as normal when it is actually normal, i.e. Pr(C(X, L)=2 |y=2).

If C(X, L) is a hard-decision classifier, we can estimate sensitivity and specificity using

$$\hat{θ} = \frac{{\sum }_{i = 1}^{n} I \{ {y}_{i} = 1 \} I \{ C ( {X}_{i} , L ) = 1 \}}{{\sum }_{i = 1}^{n} I \{ {y}_{i} = 1 \}} , \hat{η} = \frac{{\sum }_{i = 1}^{n} I \{ {y}_{i} = 2 \} I \{ C ( {X}_{i} , L ) = 2 \}}{{\sum }_{i = 1}^{n} I \{ {y}_{i} = 2 \}} .$$

sample proportions,

The most commonly used classification error (Err) is estimated as

$$\begin{matrix}\hat{Err} = \frac{{\sum }_{i = 1}^{n} I \{ C ( {X}_{i} , L ) \neq {y}_{i} \}}{n} \\ = \frac{{n}_{1}}{n} {\sum }_{i = 1}^{n} I \{ C ( {X}_{i} , L ) = 2 , {y}_{i} = 1 \} + \frac{{n}_{2}}{n} {\sum }_{i = 1}^{n} I \{ C ( {X}_{i} , L ) = 1 , {y}_{i} = 2 \} \\ = \frac{{n}_{1}}{n} ( 1 - \hat{θ} ) + \frac{{n}_{2}}{n} ( 1 - \hat{η} ) ,\end{matrix}$$

where n₁ and n₂ are sample size for cancer and normal groups. 1-θ is classification error for cancer group, and 1-η is classification error for normal group. If we have a very un-balanced sample set, i.e. n₁>>n₂ or n₁>>n₂, we can see that the previous definition of Err will encourage classifying all samples into the group with the larger sample size. To avoid this problem we can use a balanced classification error definition

$$\begin{matrix}\hat{Err} = \frac{1}{2} ( 1 - \hat{θ} ) + \frac{1}{2} ( 1 - \hat{η} ) \\ = \frac{1}{2} {\sum }_{i = 1}^{n} I \{ C ( {X}_{i} , L ) = 2 , {y}_{i} = 1 \} + \frac{1}{2} {\sum }_{i = 1}^{n} I \{ C ( {X}_{i} , L ) = 1 , {y}_{i} = 2 \} .\end{matrix}$$

This error definition assigns equal weights to two groups.

In case we have a probability output, we first select a threshold a and then define the hard-decision classifier as

$$C ( {X}_{i} , L ) = \{ \begin{matrix}1 & if {P}_{1 , i} \geq α \\ 2 & otherwise\end{matrix} .$$

We can then estimate θ, η and Err similarly as before and

$$\hat{θ ( α )} = \frac{{\sum }_{i = 1}^{n} I \{ {y}_{i} = 1 \} I \{ {P}_{t , i} \geq α \}}{{\sum }_{i = 1}^{n} I \{ {y}_{i} = 1 \}} , \hat{η ( α )} = \frac{{\sum }_{i = 1}^{n} I \{ {y}_{i} = 2 \} I \{ {P}_{t , i} < α \}}{{\sum }_{i = 1}^{n} I \{ {y}_{i} = 2 \}}$$

$$and$$

$$\hat{Err ( α )} = \frac{1}{2} ( 1 - \hat{θ ( α )} ) + \frac{1}{2} ( 1 - \hat{η ( α )} ) .$$

Relationship between \hat{θ(α)} and \hat{η(α)} is the commonly used ROC curve. Minimum classification error can be estimated as min_{αε|0,1|}\hat{Err(α)}.

Preprocessing is arguably the most important step in mass spectrometry data analysis to reduce the effects of noisy features and to appropriately interpret the mass spectrometry dataset. Before we submit the dataset to our final classifier, we carry out the following preprocessing steps: mass alignment, normalization, smoothing and peak identification. These detailed preprocessing steps are discussed briefly in Wu, B., Williams, K., and Zhao, H. Statistical challenges in proteomics research in postgenomics era. Institute of Mathematical Statistics Series IMS Lecture Notes-Monograph Series, 2003b, submitted; which is included herewith as Appendix C. Since we did not have a true test set, cross-validation was utilized to provide a nearly unbiased estimate of the classification error. The idea of cross-validation is to randomly partition the original data into two parts: training set used to build the classifier and a testing set used to estimate the performance of the classifier. The commonly used “leave-one-out” cross-validation approach has high variance. Ambroise, C., and MacLachlan, G. J. Selection bias in gene extraction on the basis of microarray gene-expression data. PNAS 99, 10 (2002), 6562-6566. M-fold cross-validation is recommended, whereby M is usually taken to be around 5, 10. In our study we use 5-fold cross-validation to estimate classification errors. It is important to carry out peak identification and biomarker selection inside each cross-validation to avoid selection bias and to obtain and unbiased classification error estimation.

It is obvious that Err depends on the underlying classifier, sample size N and the number of selected biomarkers M. In this study we fix the classifier to be RF, and evaluate the impacts of N and M on Err. Our strategy is to empirically model the functional relationship Err(N, M) for a grid of values of N, M. For mass spectrometry data the total number of features is usually very large, there are total p=130,000 m/z ratios for our ovarian cancer dataset which consists of one reflectron and one linear spectrum for each sample. The total number of selected biomarkers is usually in the range of 10˜100. In our study we evaluate Err for M ranging from 5 to 100. The total number of samples is usually very small compared to the total number of features. There are total n=170 samples in our current ovarian cancer data set. We need to extrapolate to estimate the impacts of N on Err. An inverse-power-law learning curve relationship between Err and N, Err(N)=β₀+β₁N^{-a} is approximately true for large sample size dataset (usually about tens of thousands of samples), a is the asymptotic classification error and (β₀, β₁, i) are positive constants. C. Cortes, L. D. Jackel, S. A. Solla, V. Vapnik, and J. S. Denker. Learning Curves: Asymptotic Values and Rate of Convergence. Advances in Neural Information Proceeding Systems, 6:327-334, 1994.

Our current dataset has relatively very small sample size (n=170) compared to high-dimension feature space (p=130,000 for datasets containing merged reflectron+linear analyzer spectra). Under this situation it is not appropriate to rely on the learning curve model to extrapolate to an infinite training sample size N=∞. But within a limited range we can still rely on this model to extrapolate the classification error to full sample size n=170. To estimate parameters (α, β₀, β₁), we need to obtain at least three observations. As discussed before we will use 5-fold cross-validation to estimate classification errors. We first use one of the groups as testing set, which will produce a training set of N=170/5*4=136 samples. We then use two, three and four of the groups as a testing set, which will give N=102, 68, 34. For each N we will estimate classification errors with M=5, 6, . . . , 100 biomarkers. And based on these classification errors we can estimate the learning curve.

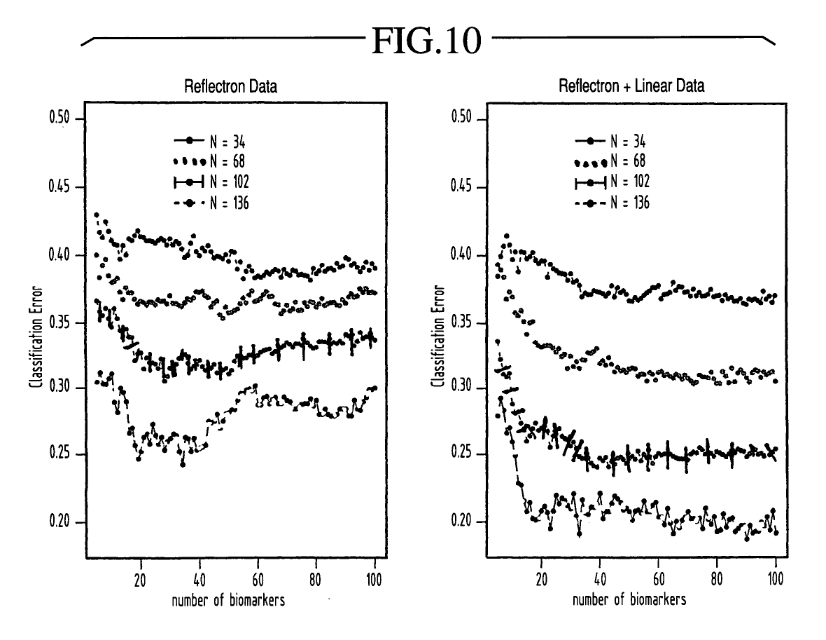

FIG. 10 displays the 5-fold cross-validation classification error estimations for this ovarian cancer data set. After merging the linear analyzer data, the best classification error achieved drops from about 25% to 20% and the classification error estimation is also more stable. The large fluctuations in classification error estimations in the Reflectron data are probably due at least in part to the influence of noise. Overall we can clearly see the trend that a larger training set has smaller classification errors. And for a fixed training set, classification error drops significantly from 5 to 20 biomarkers and then it levels off at about 20-40 biomarkers for the combined Reflectron+Linear data. With 136 samples in the training set, we can achieve about 20% classification error. Next we will use a learning curve to extrapolate Err(170, M) for each M.

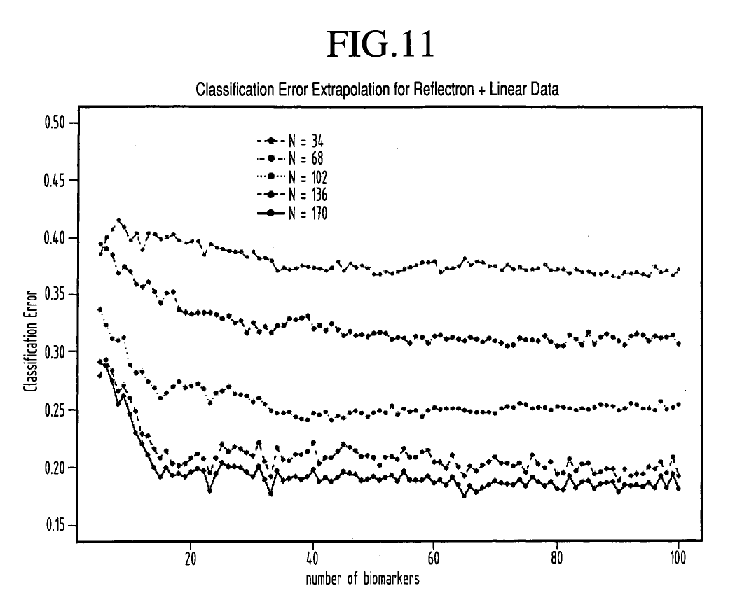

FIG. 11 displays the estimated classification for total sample size M=170. We can see that there is a significant improvement when the sample size increases from 34 to 68 and then to 102. But there is not too much further improvement from 136 samples to 170 samples. Overall the classification error levels off after 20 to 40 biomarkers. And the optimal classification error we can achieve is about 19%.

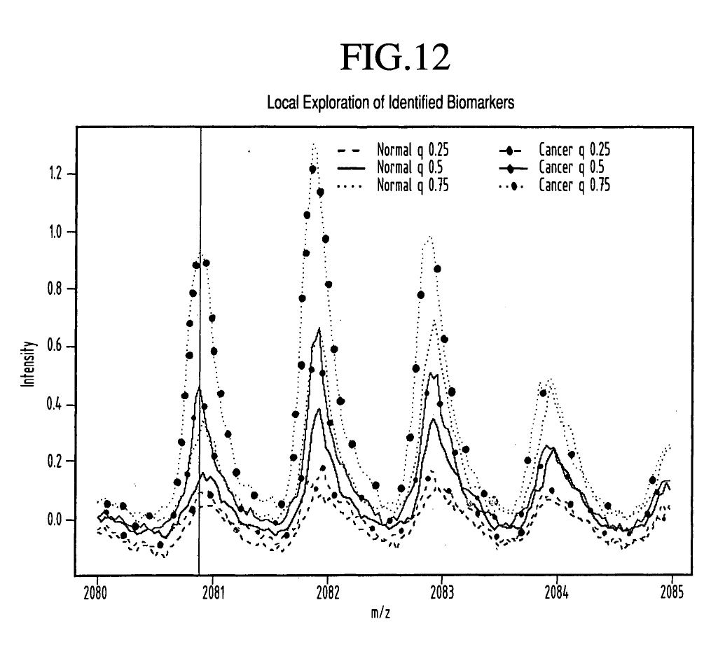

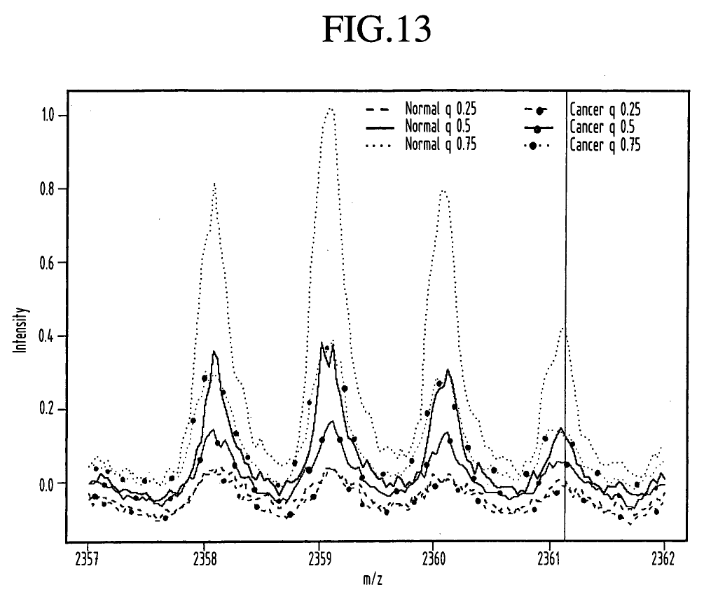

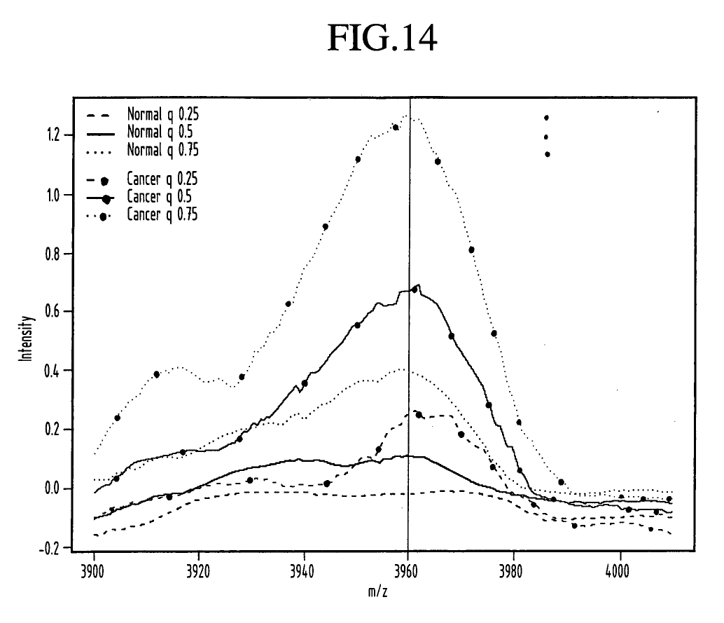

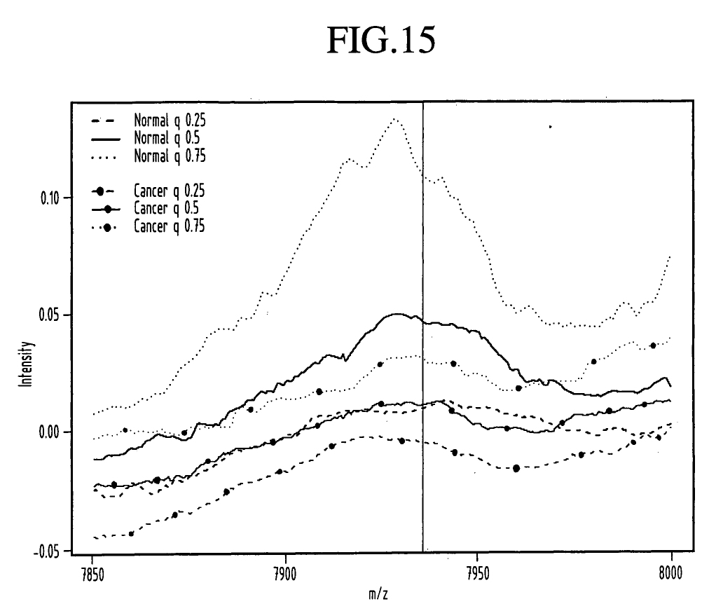

One of the major current interests in obtaining mass spectrometry data on patient samples is in identifying important biomarkers to build molecular diagnosis and prognosis tools. As discussed in Wu et al., the Random Forest program has some significant advantages over traditional T-statistic for biomarker identification in terms of minimizing classification errors. Here we apply Random Forest to our 170 ovarian cancer samples to rank important biomarkers. To guard against false positives, it is very important to explore the local behavior of the identified biomarkers. To explore the intensity of all samples in one figure will make the plot obscure. Instead we visually compare median, first and third quartile intensities of normal and cancer groups in one plot. In the following several biomarker exploration plots, q_{0.25} is the first quartile intensity, q_{0.5} the median intensity and q_{0.7} the third quartile intensity. Referring to FIGS. 12-15, we can clearly see the difference between cancer and normal groups. But there is no single biomarker that can completely distinguish cancer from normal groups; there are considerable overlaps between the two groups. For some biomarkers the normal group has higher intensities, while the cancer group dominates at other biomarkers.

We estimate the unbiased classification error rates for the ovarian cancer datasets. With reflectron data alone, we can achieve about 25% classification error. After expanding the mass range of mass spectrometry data with the use of a linear analyzer, the optimal classification error we can achieve with 170 samples is about 19% for the merged linear+reflectron spectra. While some other cancer studies using mass spectrometry data have reported nearly perfect classifications, they are usually based on internal CV that will produce serious under-estimations of the actual error, e.g. in our previous study, the optimal internal classification error is about 8% compared to the “real” classification error 25%. Wu et al. Another neglected aspect in most current studies is the lack of visualization tools to analyze the regions around the identified biomarkers and to verify that they might actually result from peptide ionization.

While the preferred embodiments have been shown and described, it will be understood that there is no intent to limit the invention by such disclosure, but rather, it is intended to cover all modifications and alternate constructions falling within the spirit and scope of the invention as defined in the appended claims.

1. A method for identification of biological characteristics, comprising the following steps:
collecting a data set relating to individuals having known biological characteristics;
analyzing the data set to identify biomarkers potentially relating to selected biological state classes.

2. The method according to claim 1, wherein the step of collecting including creating a data set of mass spectrometry spectra.

3. The method according to claim 1, wherein the step of collecting includes preprocessing of the data set.

4. The method according to claim 3, wherein the step of preprocessing includes mass alignment, normalization, smoothing and peak identification.

5. The method according to claim 3, wherein the step of preprocessing includes mass alignment.

6. The method according to claim 3, wherein the step of preprocessing includes normalization.

7. The method according to claim 3, wherein the step of preprocessing includes smoothing.

8. The method according to claim 3, wherein the step of preprocessing includes peak identification.

9. The method according to claim 1, wherein the known biological characteristic is ovarian cancer.

10. The method according to claim 1, wherein the step of analyzing is performed through application of a Random Forest algorithm.

11. The method according to claim 10, wherein the step of analyzing further includes defining sensitivity and defining specificity.

12. The method according to claim 10, wherein the selected biological state classes are no cancer and cancer.

13. The method according to claim 12, wherein the biological state class for cancer relates to ovarian cancer.

14. A system for identification of biological characteristics, comprising:
means for collecting a data set relating to individuals having known biological characteristics;
means for analyzing the data set to identify biomarkers potentially relating to selected biological state classes.

15. The system according to claim 14, wherein the means for collecting includes means for creating a data set of mass spectrometry spectra.

16. The system according to claim 15, wherein the means for collecting includes means for preprocessing of the data set.

17. The system according to claim 16, wherein the means for preprocessing includes means for mass alignment, normalization, smoothing and peak identification.

18. The system according to claim 16, wherein the means for preprocessing includes means for mass alignment.

19. The system according to claim 16, wherein the means for preprocessing includes means for normalization.

20. The system according to claim 16, wherein the means for preprocessing includes means for smoothing.

21. The system according to claim 16, wherein the means for preprocessing includes means for peak identification.

22. The system according to claim 16, wherein the known biological characteristic is ovarian cancer.

23. The system according to claim 16, wherein the means for analyzing is performed through application of a Random Forest algorithm.

24. The system according to claim 23, wherein the means for analyzing further includes means for defining sensitivity and defining specificity.

25. The system according to claim 23, wherein the means for classifying further includes means for defining sensitivity.

26. The system according to claim 23, wherein the means for classifying further includes means for defining specificity.

27. The system according to claim 23, wherein the selected biological state classes are no cancer and cancer.

28. The system according to claim 27, wherein the biological state class for cancer relates to ovarian cancer.
